SEQUENCE LISTING (4 sequences)

SEQ ID NO: 1 - DNA, 699 bp, Artificial
  Light Chain Natalizumab
  sig_peptide (1)..(54): SP(3)
atgaagtgggtgaccttcatctccctgctgtttctgttctcctccgcctactccgacatccagatgacccagtccccctccagcctgtccgcctccgtgggcgacagagtgaccatcacatgcaagacctcccaggacatcaacaagtacatggcctggtatcagcagacccccggcaaggcccctcggctgctgatccactacacctccgccctgcagcccggcatcccttccagattctccggctctggctctggccgggactacaccttcaccatctccagcctgcagcctgaggacattgccacctactactgcctgcagtacgacaacctgtggaccttcggccagggcaccaaggtggaaatcaagcggaccgtggccgctccctccgtgttcatcttcccaccctccgacgagcagctgaagtccggcaccgccagcgtggtgtgcctgctgaacaacttctacccccgcgaggccaaggtgcagtggaaggtggacaacgccctgcagagcggcaactcccaggaatccgtgaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgctgatag

SEQ ID NO: 2 - protein, 231 aa, Artificial
  Light Chain Natalizumab
  SIGNAL (1)..(18): SP(3)
  mat_peptide (19)..(231)
MKWVTFISLLFLFSSAYSDIQMTQSPSSLSASVGDRVTITCKTSQDINKYMAWYQQTPGKAPRLLIHYTSALQPGIPSRFSGSGSGRDYTFTISSLQPEDIATYYCLQYDNLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 1410 bp, Artificial
  Heavy Chain Natalizumab
  sig_peptide (1)..(54): SP(3)
atgaagtgggtgaccttcatctccctgctgtttctgttctccagcgcctactcccaggtgcagctggtgcagtctggcgccgaagtgaagaaacctggcgcctccgtgaaggtgtcctgcaaggcctccggcttcaacatcaaggacacctacatccactgggtgcgacaggcccctggccagcggctggaatggatgggcagaatcgaccccgccaacggctacactaagtacgaccccaagttccagggcagagtgaccatcaccgccgacacctccgcctccaccgcctacatggaactgtcctccctgcggagcgaggacaccgccgtgtactactgcgccagagagggctactacggcaactacggcgtgtacgccatggactactggggccagggcaccctggtgacagtgtcctccgccagcaccaagggcccctccgtgttccctctggccccttgctcccggtccacctccgagtctaccgccgctctgggctgcctggtgaaagactacttccccgagcccgtgaccgtgtcctggaactctggcgccctgacctccggcgtgcacaccttccctgccgtgctgcagtcctccggcctgtactccctgtcctccgtggtgaccgtgccatccagctccctgggcaccaagacctacacctgtaacgtggaccacaagccctccaacaccaaggtggacaagcgggtggaatctaagtacggccctccctgccccagctgccctgcccctgaattcctgggcggaccttccgtgttcctgttccccccaaagcccaaggacaccctgatgatctcccggacccccgaagtgacctgcgtggtggtggacgtgtcccaggaagatcccgaggtgcagttcaattggtacgtggacggcgtggaagtgcacaacgccaagaccaagcccagagaggaacagttcaactccacctaccgggtggtgtctgtgctgaccgtgctgcaccaggactggctgaacggcaaagagtacaagtgcaaggtgtccaacaagggcctgcccagctccatcgaaaagaccatctccaaggccaagggccagccccgcgagccccaggtgtacaccctgccccctagccaggaagagatgaccaagaaccaggtgtccctgacctgtctggtgaaaggcttctacccctccgacattgccgtggaatgggagtccaacggccagcccgagaacaactacaagaccaccccccctgtgctggactccgacggctccttcttcctgtactctcggctgaccgtggacaagtcccggtggcaggaaggcaacgtgttctcctgctccgtgatgcacgaggccctgcacaaccactacacccagaagtccctgtccctgagcctgggcaagtgatag

SEQ ID NO: 4 - protein, 468 aa, Artificial
  Heavy Chain Natalizumab
  SIGNAL (1)..(18): SP(3)
  mat_peptide (19)..(468)
MKWVTFISLLFLFSSAYSQVQLVQSGAEVKKPGASVKVSCKASGFNIKDTYIHWVRQAPGQRLEWMGRIDPANGYTKYDPKFQGRVTITADTSASTAYMELSSLRSEDTAVYYCAREGYYGNYGVYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2019/0374639 A1
(43) Pub. Date: Dec. 12, 2019

(54) AQUEOUS PHARMACEUTICAL FORMULATIONS

(71) Applicant: POLPHARMA BIOLOGICS S.A., Gdansk (PL)

(72) Inventors: Dariusz Mateusz Suchy, Gdansk (PL); Malgorzata Bozena Tracka, Borkowo (PL); Kornelia Bogumila Wisniewska, Gdansk (PL); Michael Josef Safinowski, Breitenbach am Inn (AT); Klemen Spaninger, Gdansk (PL); Piotr Marcin Zien, Sadowa (PL)

(21) Appl. No.: 16/462,827

(22) PCT Filed: Nov. 21, 2017
(86) PCT No.: PCT/EP2017/079891

§ 371 Date: May 21, 2019

(30) Foreign Application Priority Data
Nov. 21, 2016 (EP) 16460087.6

(51) Int. Cl. A61K 39/395; C07K 16/28; A61K 47/22; A61K 47/02; A61K 47/26; A61K 47/18

(52) U.S. Cl. CPC A61K 39/39591; C07K 16/2842; A61K 47/22; C07K 2317/24; A61K 47/26; A61K 47/183; A61K 47/02

(57) ABSTRACT

The present invention belong to the field of biotechnology, in particular to the field of galenics. The present invention is directed to an aqueous pharmaceutical composition comprising 1-50 mg/ml of a human or humanized full length IgG₄, e.g. natalizumab, and a formulation selected from (i) to (iii), as further defined in the claims. The present invention is further directed to a method for producing said pharmaceutical composition, and medical uses thereof. Finally, also provided is a method for stabilizing human or humanized full length IgG₄, e.g. natalizumab, as further defined in the claims.

The present invention is directed to an aqueous pharmaceutical composition comprising 1-50 mg/ml of a human or humanized full length IgG₄, e.g. natalizumab, and a formulation selected from (i) to (iii), as further defined in the claims. The present invention is further directed to a method for producing said pharmaceutical composition, and medical uses thereof. Finally, also provided is a method for stabilizing human or humanized full length IgG₄, e.g. natalizumab, as further defined in the claims.

### BACKGROUND OF THE INVENTION

There are four different subclasses of IgG antibodies, IgG₁ to IgG₄, which differ in their sequence and function. For example, IgG₃ is characterized by a longer and more flexible hinge domain and the presence of 11 inter-heavy chain disulfide bridges, while IgG₁ and IgG₄ comprises only 2, and IgG₂ comprises 4 inter-heavy chain disulfide bridges. Apart thereof, IgG₄ antibodies are secreted as both, disulfide-linked tetramers and half-molecules, linked by strong non-covalent interactions, which is not observed in other IgG subclasses. Moreover, IgG₄ can undergo Fab-arm exchange. The particularities of IgG₄ structure and function are reviewed, for example, in Davies & Sutton, Immunol. Rev. 268(1): 139-159 (2015). As a consequence, requirements for a stable aqueous pharmaceutical composition for human or humanized IgG₄ antibodies differ from those reported for other human IgG subclasses such as IgG₁, IgG₂ and IgG₃.

The recombinant therapeutic monoclonal antibody natalizumab is an IgG₄ full-length antibody humanized from a murine monoclonal antibody that binds to the α₄β₁ integrin (also known as VLA-4 or CD49d-CD29) and α₄β₇ integrin, and blocks the interaction of said α₄ integrins with their respective receptors VCAM-1 and MadCAM-1 which are expressed on endothelial cells. See also WO 95/19790. α₄-integrin is required for inflammatory lymphocytes to attach to and pass through the cell layers lining the intestine and blood-brain-barrier.

Natalizumab is marketed by Biogen MA Inc. under the name Tysabri, and was previously named Antegren. It has FDA-approval for the treatment of multiple sclerosis and Crohn's disease, and EMEA approval for the treatment of multiple sclerosis. Recently, it was suggested that natalizumab could also be used in a combination treatment of B-cell malignancies, where it is intended to overcome the resistance to rituximab. Natalizumab is typically administered by intravenous infusion. According to the Scientific Discussion available from the EMEA, natalizumab is recombinantly produced in a NS/0 murine myeloma cell line. The antibody is then purified using Protein A affinity chromatography and hydrophobic interaction chromatography, followed by a buffer exchange and concentration by ultrafiltration/diafiltration.

In the current commercialized formulation, natalizumab is simply formulated in a PBS-Tween standard buffer comprising 10 mM sodium phosphate, 140 mM sodium chloride, 0.02% (w/v) polysorbate 80, pH 6.1, which does not comprise a polyol. See also WO 2004/071439 A1, EP 2 236 154 A1, and the preferred embodiments therein (e.g. claim 34 of WO 2004/071439 A1). WO 2004/071439 A1 further discloses a solution for lyophilisation of 20 mg/ml natalizumab comprising 5 mM histidine, 20 mg/ml sucrose, 0.02% polysorbate 80 at pH 6.0 (cf. claim 40).

WO 2014/160490 discloses antibody formulations comprising 15-100 mg/ml monoclonal antibody, 15-35 mM phosphate buffer, pH 5.5-7.0, 0.01%-0.1% surfactant and trehalose in an amount of up to 2.7% (w/v).

Spiess et al. Journal of Biological Chemistry 288(37): 26583-26593 (2013) disclose IgG₄ antibody formulations comprising 45 mg/ml antibody, 0.2 M arginine succinate, pH 5.5, 0.02% polysorbate 20.

EP 1 314 437 A1 discloses formulations of humanized IgG₁ antibody hPM-1. While EP 1 314 437 A1 teaches the use of formulations comprising 1-50 mM histidine at a pH of 5.5-6.2, preferred embodiments comprise 5-10 mM histidine at a pH of 6.2-7.5. According to paragraph [0062] of EP 1 314 437 A1, addition of sodium chloride resulted in increased aggregation, for which reason it is taught that these formulations shall not contain sodium chloride (cf. claim 8 of EP 1 314 437 A1).

WO 98/56418 A1 discloses pharmaceutical compositions of the humanized IgG₁ antibodies H52 (directed against CD18) and C2B8 (directed against CD20). While initially, histidine 10 mM histidine buffer buffer, 140 mM sodium chloride, 0.01% Tween-20, pH 6.0 is tested, the authors come to the conclusion that it is more desirable to use a sugar as the tonicifier, rather than a salt, so as to minimize oxidation at low pH (cf. page 27, lines 8-9). The finally preferred formulation is set out in the claims of WO 98/56418 A1, and comprises histidine or acetate buffer in combination with a polyol, and polysorbate, pH 4.5-6.0. The most preferred embodiment of claim 21 comprises 20-30 mM acetate buffer at about pH 5, 1-15% (w/v) trehalose, 0.01-0.03% polysorbate, and 0.5 to 1% benzyl alcohol.

U.S. Pat. Nos. 8,420,081 and 8,883,146 describe pharmaceutical compositions for human IgG₁ 1D4.7 and 13C5.5 having a conductivity of less than 2.5 mS/cm. The pharmaceutical compositions of the present disclosure are neither described nor suggested.

It is thus the object of the present invention to provide pharmaceutical compositions of human or humanized IgG₄. Another object of the present invention is to provide pharmaceutical compositions for human or humanized IgG₄ which can be used as alternatives to formulations known from the prior art.

The present invention demonstrates that the stability can be maintained by formulating human or humanized IgG₄ in the pharmaceutical compositions of the present disclosure. The pharmaceutical compositions of the present invention protect human or humanized IgG₄ against degradation induced by temperature stress or mechanical stress. The increased physical stability of the pharmaceutical composition results in a long shelf-life and ensures product safety.

### SUMMARY OF THE INVENTION

The present inventors have conducted a research programme and found aqueous pharmaceutical compositions for a human or humanized full length IgG₄ antibody, which exhibits advantageous properties, e.g. a good stability and thereby a long shelf-life. In case of natalizumab, the formulations show a stability comparable to the commercial formulation. In particular, it is provided an aqueous pharmaceutical composition comprising 1-50 mg/ml of a human or humanized full length IgG₄, and a formulation selected from
(i) a formulation comprising 10-25 mM histidine buffer, 130-180 mM sodium chloride, and 0.001-0.5% (w/v) of a surfactant, pH 5.1-6.5; or
(ii) a formulation comprising 25-30 mM phosphate buffer, 5-10% (w/v) of a polyol, and 0.001-0.5% (w/v) of a surfactant, pH 5.0-6.9; or
(iii) a formulation comprising 10-25 mM histidine buffer, 10-50 mM sodium chloride, and 0.001-0.5% (w/v) of a surfactant, pH 5.1-6.5.

In a preferred embodiment, the human or humanized full-length IgG₄ is natalizumab. Further provided is an article of manufacture, comprising a pharmaceutical composition as described herein and instructions for use of said composition, preferably wherein said pharmaceutical composition is contained in a pre-filled vial or in a pre-filled syringe, more preferably wherein said pharmaceutical composition is contained in a pre-filled vial.

In addition, a method for producing an aqueous pharmaceutical composition as described herein is provided, the method comprising combining 1-50 mg/ml of a human or humanized full-length IgG₄ with a formulation selected from
(i) a formulation comprising 10-25 mM histidine buffer, 130-180 mM sodium chloride, and 0.001-0.5% (w/v) of a surfactant, pH 5.1-6.5; or
(ii) a formulation comprising 25-30 mM phosphate buffer, 5-10% (w/v) of a polyol, and 0.001-0.5% (w/v) of a surfactant, pH 5.0-6.9; or
(iii) a formulation comprising 10-25 mM histidine buffer, 10-50 mM sodium chloride, and 0.001-0.5% (w/v) of a surfactant, pH 5.1-6.5.

Also provided is a method for stabilizing a human or humanized full length IgG₄, comprising combining 1-50 mg/ml of a human or humanized full length IgG₄ with a formulation selected from
(i) a formulation comprising 10-25 mM histidine buffer, 130-180 mM sodium chloride, and 0.001-0.5% (w/v) of a surfactant, pH 5.1-6.5; or
(ii) a formulation comprising 25-30 mM phosphate buffer, 5-10% (w/v) of a polyol, and 0.001-0.5% (w/v) of a surfactant, pH 5.0-6.9; or
(iii) a formulation comprising 10-25 mM histidine buffer, 10-50 mM sodium chloride, and 0.001-0.5% (w/v) of a surfactant, pH 5.1-6.5, thereby preparing a stable aqueous pharmaceutical composition.

The stable aqueous pharmaceutical composition described herein is particularly useful in the treatment of multiple sclerosis or Crohn's disease.

### DETAILED DESCRIPTION OF THE PREFERRED EMBODIMENTS

More specifically, the present disclosure provides an aqueous pharmaceutical composition comprising, preferably consisting of, 1-50 mg/ml of a human or humanized full length IgG₄, and a formulation selected from
(i) a formulation comprising 10-25 mM histidine buffer, 130-180 mM sodium chloride, and 0.001-0.5% (w/v) of a surfactant, pH 5.1-6.5; or
(ii) a formulation comprising 25-30 mM phosphate buffer, 5-10% (w/v) of a polyol, and 0.001-0.5% (w/v) of a surfactant, pH 5.0-6.9; or
(iii) a formulation comprising 10-25 mM histidine buffer, 10-50 mM sodium chloride, and 0.001-0.5% (w/v) of a surfactant, pH 5.1-6.5.

Among the four formulations (i)-(iii), formulation (i) is particularly preferred.

The aqueous pharmaceutical composition described herein is not intended for lyophilisation, and has not been previously lyophilized. In other words, the pharmaceutical composition described herein is intended to be stored in “dissolved” form, as also apparent from the indicated concentration ranges and the term “aqueous”. In a preferred embodiment, the pharmaceutical composition is in liquid form.

The aqueous pharmaceutical composition described herein are expected to be stable.

The term “stable” as used herein is intended to mean that the composition retains its physical stability, and/or conformational stability and/or colloidal stability upon storage, as further described below. The composition may also retain chemical stability and/or biological activity, as further described below.

The term “physical stability” refers to the ability of a product to maintain its physical dimensions when exposed to conditions normally encountered in its service environment. In particular, the pharmaceutical composition described herein exhibits a comparable physical stability to the same human or humanized IgG₄ antibody formulated at the same concentration in 10 mM sodium phosphate, 140 mM sodium chloride, 0.02% (w/v) polysorbate 80, pH 6.1. Physical stability can be measured in terms of precipitation, degradation, and/or denaturation, and can be measured, for example, by visual inspection of color and/or clarity, size exclusion chromatography, or micro flow imaging, the determination and assessment of which is further described in the examples below.

In another preferred embodiment, the pharmaceutical composition exhibits less sub-visible particles (>10 μm) as compared to the same human or humanized IgG₄ antibody formulated at the same concentration in 10 mM sodium phosphate, 140 mM sodium chloride, 0.02% (w/v) polysorbate 80, pH 6.1 after three cycles of freezing from room temperature to −80° C. for 8 h and then thawing for 2 h at room temperature. In a more preferred embodiment, the pharmaceutical composition exhibits less than 99% sub-visible particles, preferably less than 95%, more preferably less than 90%, more preferably less than 85%, more preferably less than 80%, more preferably less than 75%, even more preferably less than 70%, and most preferably less than 65% sub-visible particles (>10 μm) as determined by microflow imaging after three cycles of freezing from room temperature to −80° C. for 8 h and then thawing for 2 h at room temperature. Microflow imaging is a technique that allows to characterize sub-visible particles in the range of 2 μm to 200 μm in a given formulation. Before sample preparation, the system is primed with related formulation buffer to optimize illumination. Samples are then prepared by gently mixing in circular motions after removal from storage conditions. Then the vials are opened and 1 ml of sample is withdrawn and loaded in the inlet port of the microflow imaging system. Data is acquired using the following parameters: Sample dispensed: 0.9 ml; purge volume: 0.2 ml; sample analyzed volume: 0.59 ml; edge particles rejection: enabled; fill particles: enabled; and the data is analyzed by setting filters above 10 μm. Preferably the samples are neat at target concentration of about 20 mg/ml. Further guidance is provided in the examples below.

In another preferred embodiment, the pharmaceutical composition exhibits less than 1% aggregation products, in particular less than 0.95%, such as less than 0.9%, or even less than 0.85%, as determined after four weeks of storage at 40° C. by size exclusion chromatography.

In addition or alternatively, the pharmaceutical composition exhibits an increase of less than 0.5% of aggregation products, in particular less than 0.45%, such as less than 0.4%, or even less than 0.35%, as determined after four weeks of storage at 40° C. by size exclusion chromatography. Certain formulations of the present disclosure even exhibit less than 0.5%, in particular less than 0.4% such as less than 0.35% aggregation products as determined after four weeks of storage at 40° C. by size exclusion chromatography.

In addition, the pharmaceutical composition exhibits less aggregation products as compared to the same human or humanized IgG₄ antibody formulated at the same concentration in 10 mM sodium phosphate, 140 mM sodium chloride, 0.02% (w/v) polysorbate 80, pH 6.1, as determined after at four weeks of storage at 40° C., 25° C., or 5° C., preferably at 40° C., by size exclusion chromatography. In a more preferred embodiment, the pharmaceutical composition exhibits less than 99% aggregation products, preferably less than 98.5%, more preferably less than 98%, even more preferably less than 97.5% aggregation products, and most preferably less than 97% aggregation products as determined after four weeks of storage at 40° C., 25° C., or 5° C., preferably at 40° C., by size exclusion chromatography.

In addition, or alternatively, the pharmaceutical composition may exhibit less degradation products as compared to the same human or humanized IgG4 antibody formulated at the same concentration in 10 mM sodium phosphate, 140 mM sodium chloride, 0.02% (w/v) polysorbate 80, pH 6.1 as determined after storage for 12, preferably 20, more preferably 24, more preferably 48, more preferably 72, and most preferably 96 weeks at 40° C. by size exclusion chromatography. In a more preferred embodiment, the pharmaceutical composition may exhibit less than 99% degradation products, preferably less than 98.5%, more preferably less than 98%, even more preferably less than 97.5% degradation products, and most preferably less than 97% degradation products as determined after storage for 12, preferably 20, more preferably 24, more preferably 48, more preferably 72, and most preferably 96 weeks at 40° C. by size exclusion chromatography. Size exclusion chromatography is performed using UPLC system equipped with Waters BEH200 SEC 1.7 um 4.6×150 mm at column temperature 30±0.5° C. The mobile phase is 100 mM sodium phosphate buffer with 200 mM sodium chloride, pH 6.8±0.05 at a flow rate of 0.3 ml/min. The samples are diluted to 1 mg/ml in the respective buffer and then a volume of 2 μl is injected to the system. Detection is carried out at 210 nm.

Preferably, the pharmaceutical composition exhibits less than 3% of IgG₄ half antibodies, in particular less than 2.9%, preferably less than 2.5%, more preferably less than 2.2%, and most preferably less than 2.1%, after storage for four weeks at 40° C., as determined by polyacrylamide gel electrophoresis under non-reducing conditions and colloidal Coomassie Blue gel staining. Polyacrylamide gel electrophoresis under non-reducing conditions and colloidal Coomassie Blue gel staining may be carried out using the Experion® electrophoresis system (Biorad) as described in Example 3 below.

In another preferred embodiment, the pharmaceutical composition exhibits less percent half antibodies as compared to the same human or humanized IgG₄ antibody formulated at the same concentration in 10 mM sodium phosphate, 140 mM sodium chloride, 0.02% (w/v) polysorbate 80, pH 6.1, after storage for four weeks at 40° C., as determined by polyacrylamide gel electrophoresis under non-reducing conditions and colloidal Coomassie Blue gel staining. Polyacrylamide gel electrophoresis under non-reducing conditions and colloidal Coomassie Blue gel staining may be carried out using the Experion® electrophoresis system (Biorad) as described in Example 3 below.

The term “conformational stability” refers to the denaturation temperature and denaturation enthalpy of proteins and other macromolecules in solution as a measure of molecular stability in a given environment. Conformational stability can be measured in, for example, by Differential Scanning Calorimetry (DSC). DSC measures the amount of heat that is absorbed or released from biomolecules in solution during heating. Native proteins respond to heating by unfolding (thermal denaturation) at a characteristic temperature (Tm). The more intrinsically stable the biopolymer is the higher is the midpoint temperature of the unfolding transition (Tm) and the transition enthalpy. Measurements are carried out in scanning mode in a temperature range of 20° C. to 100° C. with heating and scanning rate of 1° C./min; a buffer scan is conducted prior to each sample run to generate a baseline. Samples are prepared via dialysis against the corresponding formulation buffer and diluted to about 1 mg/ml. The corresponding dialysis buffer is used as buffer scan. The exact sample concentration after dialysis is determined by UV (280 nm) absorption.

In a preferred embodiment, the human or humanized IgG₄ have their Tₘ₁ (CH₂ domain unfolding) in the range from 65° C. to 70° C., as determined by DSC. In another preferred embodiment, the pharmaceutical composition described herein exhibits a higher conformational stability as compared to the same human or humanized IgG₄ antibody formulated at the same concentration in 10 mM sodium phosphate, 140 mM sodium chloride, 0.02% (w/v) polysorbate 80, pH 6.1. Even more preferably, the Tm is more than 0.1° C. higher in the pharmaceutical composition described herein as compared to the same human or humanized IgG₄ antibody formulated at the same concentration in 10 mM sodium phosphate, 140 mM sodium chloride, 0.02% (w/v) polysorbate 80, pH 6.1; more preferably the Tₘ is more than 0.25° C., more preferably more than 0.5° C., more preferably more than 0.75° C., more preferably more than 1.0° C., even more preferably more than 1.25° C., still more preferably more than 1.5° C., and most preferably more than 1.75° C. higher; in particular after three cycles of freezing from room temperature to −80° C. for 8 h and then thawing for 2 h at room temperature.

The term “colloidal stability” refers to the molecules' interaction in Newtonian solutions, as determinable by Composition gradient multi-angle light scattering (CG-MALS). An interaction between protein molecules in solution is characterized by changes in their light scattering behavior at different concentrations via CG-MALS. The characteristic parameter to measure molecule interactions is the second virial coefficient A₂. A₂ is characteristic for a macromolecule and its solvent, and describes molecular interactions between the dissolved macromolecules. A negative A₂ indicates attractive interactions whereas a positive A₂ is characteristic for repulsive interactions between the dissolved molecules. The CG-MALS technique is based on a concentration series of a macromolecular solution which is directly injected into the flow cell of a multi angle light scattering detector. After each injection the flow is stopped to permit the reaction to reach equilibrium. The apparent molecular weight (Mwₐₚₚ) is determined for each step in the gradient by analyzing light scattering and concentration data by the following equation:

$${Mw}_{app} \vert = \frac{R ( θ , c )}{K \times c}$$

wherein
Mwₐₚₚ: apparent molecular weight
R(θ,c): excess Rayleigh ratio of the solution as a function of scattering angle θ and concentration c. It is directly proportional to the intensity of the excess light scattered by the solute and the light scattered by the pure solvent.
K: Optical constant (4p²(dn/dc)²n₀²/NₐI₀⁴)

### c: Concentration

Significant interactions between macromolecules manifest as changes in Mwₐₚₚ VS. concentration.

A2 calculation is then conducted via Zimm plot analysis according to:

$$\frac{K \cdot c}{R ( θ , c )} = \frac{1}{{M}_{w} P ( θ )} + {A}_{2} c$$

wherein
R(θ,c): excess Rayleigh ratio of the solution as a function of scattering angle θ and concentration c. It is directly proportional to the intensity of the excess light scattered by the solute and the light scattered by the pure solvent.
Mw: Weight average molecular weight
A₂: 2^{nd} virial coefficient

### c: Concentration

K: Optical constant (4p²(dn/dc)²n₀²/NₐI₀⁴)
P(θ): describes the angular dependence of the scattered light, and can be related to the rms radius. Below rms radii of 30 nm this term becomes 0.

In a preferred embodiment, the pharmaceutical composition described herein exhibits a higher colloidal stability as compared to the same human or humanized IgG₄ antibody formulated at the same concentration in 10 mM sodium phosphate, 140 mM sodium chloride, 0.02% (w/v) polysorbate 80, pH 6.1, as determined by the calculation of A₂. Even more preferably, the A₂ of the pharmaceutical composition is higher than −0.125, more preferably higher than −0.120, more preferably higher than −0.115, even more preferably higher than −0.110, and most preferably higher than −0.105.

The term “chemical stability” as used herein refers to chemical alterations of the human or humanized IgG₄. Chemical alterations include charge alteration (e.g. occurring as results of deamidation) and oxidation of certain amino acids in the sequence, but also includes degradation of the protein at several independent sites. Such modifications may result in the presence of many different species in the final product. Monoclonal antibodies therefore display considerable heterogeneity that can be characterized by ion exchange liquid chromatography (IEX-LC). In a preferred embodiment, the separation is carried out by Cation Exchange Ultra Performance Liquid Chromatography on UPLC H-Class Bio System using UV detection under Empower™ Software control. The Waters Protein-Pak Hi Res SP (7 μm, 4.6 mm i.d.×100 mm) is used for testing applying a linear gradient of NaCl. Eluents were: buffer A (14 mM NaPi buffer pH 6.0) and buffer B (10.5 mM NaPi buffer pH 6.0, 0.125 M NaCl). Gradient starts with pre-equilibration of 100% buffer A in 2 min. Elution gradient starts from 10% to 30% of buffer B in 25 min, followed by a second elution step for 5 min at 30% to 60% B and re-equilibration with 90% solvent A. The total run time is 40 min. The flow rate is 0.7 ml/min. The column temperature is 40° C. and elution is monitored at 220 nm. For data evaluation Waters Empower 3 software is used. The peak assignment is performed by retention time. The sample composition is determined by detecting peaks based on their retention time and the relative proportions of each peak are calculated from the peak areas. The final results are presented as a sum of acidic species, main peak and sum of basic species. Exemplary results of charge variants content for a reference product (range based on 20 batches testing) are presented in the following table:

| | CEX_MAIN PEAK | | | | | |
|---|---|---|---|---|---|---|
| Sample | rep. No (n) | Average Results | min | max | SD | CV |
| Referent | 1 | 71.3 | 69.0 | 72.7 | 0.993 | 1.4 |
| (20 batches) | | | | | | |

| | CEX_SUM of ACIDIC PEAKS | | | | | |
|---|---|---|---|---|---|---|
| | rep. No | | | | | |
| Sample | (n) | Average Results | min | max | SD | CV |
| Referent | 1 | 13.0 | 10.3 | 15.3 | 1.393 | 10.7 |
| (20 batches) | | | | | | |

| | CEX_SUM of BASIC PEAKS | | | | | |
|---|---|---|---|---|---|---|
| Sample | rep. No (n) | Average Results | min | max | SD | CV |
| Referent | 1 | 15.7 | 13.6 | 18.5 | 1.453 | 9.2 |
| (20 batches) | | | | | | |

The human or humanized IgG₄ may have a higher chemical stability in the pharmaceutical composition upon storage for 12, preferably 20, more preferably 24, more preferably 48, more preferably 72, and most preferably 96 weeks at 5° C.±3° C. as compared to the same human or humanized IgG₄ antibody formulated at the same concentration in 10 mM sodium phosphate, 140 mM sodium chloride, 0.02% (w/v) polysorbate 80, pH 6.1, as determined by ion exchange liquid chromatography (IEX-LC). The main peak may comprise more than 73%, preferably more than 75%, more preferably more than 80%, even more preferably more than 85%, and most preferably more than 90% upon storage for 20, preferably 24, more preferably 48, more preferably 72, and most preferably 96 weeks at 5° C.±3° C. In addition, or alternatively, the sum of acidic peaks may comprise less than 14.5%, preferably less than 14%, more preferably less than 13.5%, more preferably less than 13%, even more preferably less than 12.5%, and most preferably less than 12% upon storage for 12, preferably 20, more preferably 24, more preferably 48, more preferably 72, and most preferably 96 weeks at 5° C.±3° C. In addition, or alternatively, the sum of basic peaks may comprise less than 12.5%, preferably less than 12%, more preferably less than 11.5%, more preferably less than 11%, even more preferably less than 10.5%, and most preferably less than 10% upon storage for 12, preferably 20, more preferably 24, more preferably 48, more preferably 72, and most preferably 96 weeks at 5° C.±3° C.

The term “biological stability” means that the human or humanized IgG4 retains a higher biological activity in the pharmaceutical composition upon storage as compared to the to the same human or humanized IgG4 antibody formulated at the same concentration in 10 mM sodium phosphate, 140 mM sodium chloride, 0.02% (w/v) polysorbate 80, pH 6.1, as determined in a relevant antigen binding assay, as those below.

Mechanism of action for natalizumab involves blocking interaction of α₄β₁ and α₄β₇ integrins with their cognate receptors VCAM-1 and MadCAM-1, respectively. For example, biologic activity of natalizumab may be tested by a direct ELISA detecting binding of natalizumab to α₄β₁ integrin. The principle of this method is to incubate a coated constant amount of integrin α₄β₁ with serially diluted natalizumab samples. The amount of bound natalizumab is subsequently determined by a mouse, monoclonal anti-human IgG antibody, which is conjugated to horseradish peroxidase (HRP). HRP converts the chromogenic substrate TMB (3,3′,5,5′-tetramethylbenzidine) into a colored dye. The color reaction is measured spectrophotometrically at wavelength 450 nm. Data are analyzed applying 4 Parameter Logistic nonlinear regression model (4PL), which is commonly used for curve-fitting analysis in bioassays or immunoassays such as ELISAs or dose-response curves. Final result is expressed as a Relative Potency (REP) of tested sample in relation to interim reference standard established at Polpharma site. The method variability was determined at the level of 7% coefficient variation (CV) of intermediate precision within the qualification exercise.

Alternatively, one can also measure VCAM-1 competitive binding by ELISA. The aim of this assay is to test the ability of natalizumab to inhibit interaction of α₄β₁ integrin with its cognate receptor—VCAM-1 protein in a dose-dependent manner. Constant amount of the coated VCAM-1 protein is incubated with serial dilutions of natalizumab in the presence of HIS-tagged α₄β₁ integrin. Solid-phase associated VCAM-1 and soluble natalizumab now compete for binding to α₄β₁ integrin. The higher the natalizumab concentration the more α4β1 integrin is inhibited from binding to VCAM-1. The highest signal result is observed when no natalizumab is present. Bound HIS-tagged α₄β₁ integrin is subsequently detected with a biotinylated anti-HIS-tag antibody, POD-conjugated Streptavidin and a TMB-substrate reaction at the end of the assay. Data are analyzed with 4PL fitting model. Final result is expressed as a Relative Potency (REP) of tested sample in relation to reference standard. The method variability was determined at the level of 7% coefficient variation (CV) of intermediate precision within the qualification exercise. Additionally accuracy, linearity and specificity were tested.

| | VCAM-1 competitive ELISA | | | | | |
|---|---|---|---|---|---|---|
| | rep. No | | | | | |
| Sample | (n) | Average Results | min | max | SD | CV |
| Referent | 3 | 1.044 | 0.916 | 1.200 | 0.081 | 7.74 |
| (26 batches) | | | | | | |

In still another alternative one can measure MadCAM-1 competitive binding by ELISA. The aim of this assay is to test the ability of natalizumab to inhibit interaction of α₄β₇ integrin with its cognate receptor—MadCAM-1 protein in a dose-dependent manner. Constant amount of the coated α₄β₇ integrin is incubated with serial dilutions of natalizumab in the presence of Fc-tagged MadCAM-1 receptor. Natalizumab and MadCAM-1 receptor now compete for binding to solid-phase associated α₄β₇ integrin. The higher the natalizumab concentration the more MadCAM-1 is inhibited from binding to α₄β₇ integrin. The lowest signal result is observed when no natalizumab is present. Bound natalizumab is subsequently detected with a POD-conjugated anti-human IgG antibody and a TMB-substrate reaction at the end of the assay. Data are analyzed with 4PL fitting model. Final result is expressed as a Relative Potency (REP) of tested sample in relation to reference standard. The method variability was determined at the level of 8% coefficient variation (CV) of intermediate precision within the qualification exercise. Additionally accuracy, linearity and specificity were tested.

| | MadCAM-1 competitive ELISA | | | | | |
|---|---|---|---|---|---|---|
| | rep. No | | | | | |
| Sample | (n) | Average Results | min | max | SD | CV |
| Referent | 3 | 0.995 | 0.830 | 1.170 | 0.072 | 7.27 |
| (24 batches) | | | | | | |

The biological activity of the antibody may be at least 80% of the biological activity before storage for 12, preferably 20, more preferably 24, more preferably 48, more preferably 72, and most preferably 96 weeks at 5° C.±3° C. (within error of the assay), more preferably at least 82%, more preferably at least 84%, more preferably at least 86%, more preferably at least 88%, more preferably at least 90%, more preferably at least 92%, more preferably at least 94%, even more preferably at least 96%, still more preferably at least 98%, and most preferably at least 99% of the original activity before storage for 12, preferably 20, more preferably 24, more preferably 48, more preferably 72, and most preferably 96 weeks at 5° C.±3° C.

As exemplified below, stability of pharmaceutical compositions according to the present disclosure are assessed during a 96 weeks-stability study at intended (5° C.) as well as accelerated storage condition (25 and 40° C.). In view of the results, the pharmaceutical formulation described herein is expected to be suitable for long term storage. As used herein, the term “long term storage” shall refer to storage of a composition comprising the pharmaceutical formulation for more than 4 months, preferably for more than 12, 20, 24, 48, 72 or even 96 weeks at 5° C.±3° C.

The pharmaceutical composition of the present disclosure comprises 1-50 mg/ml of a human or humanized full-length IgG₄. In a preferred embodiment, said pharmaceutical composition comprises 5-50 mg/ml, preferably 10-45 mg/ml, more preferably 15-40 mg/ml, even more preferably 20-30 mg/ml of said human or humanized full-length IgG₄. In a most preferred embodiment, the pharmaceutical composition of the present disclosure comprises 20 mg/ml of said human or humanized full-length IgG₄.

The term “human or humanized full length IgG₄” as used herein means a full length immunoglobulin comprising the sequence of the constant region of the heavy chain of human IgG₄, which is well-known in the field, e.g. from Uniprot database entry P01861, and the constant region of a human light chain. The variable region of the heavy and light chain preferably also each comprise a human framework, while the CDR regions may originate from human or another species such as mouse. In a particular embodiment, the IgG₄ is a humanized IgG₄, in which the CDRs have been grafted in a human framework. Such human framework sequences of the variable domains are known in the art, and further exemplified in SEQ ID NOs 2 and 4 herein. In such humanized IgG₄ antibodies, it might be necessary that a limited number of amino acid residues (less than 10, preferably less than 9, more preferably less than 8, more preferably less than 7, more preferably less than 6, more preferably less than 5, more preferably less than 4, more preferably less than 3, more preferably less than 2, and most preferably no more than one amino acid residue) are/is mutated in the human IgG₄ sequence, in order to improve stability and/or better integrate the grafted sequences into the human IgG₄ framework. To that end, it was recently reported that the point substitution S228P in the hinge region prevents half molecule exchange of IgG₄, and thus further stabilizes the IgG₄ molecule (Silva et al., J Biol Chem. 290(9): 5462-5469 (2015)). Examples of therapeutic humanized IgG₄ antibodies, are Natalizumab, Gemtuzumab, and Pembrolizumab.

In a still more preferred embodiment, the human or humanized full length IgG₄ is essentially identical/similar to natalizumab, most preferably, the human or humanized full length IgG₄ is natalizumab. Natalizumab is a full length humanized IgG₄ antibody with an amino acid sequence shown in amino acids 19 to 231 of SEQ ID NO: 2 and amino acids 19 to 468 of SEQ ID NO: 4. The term “essentially identical/similar to natalizumab” as used herein means that the amino acid sequence of the human or humanized IgG₄ has at least 95% identity to the amino acid sequence shown in amino acids 19 to 231 of SEQ ID NO: 2 and amino acids 19 to 468 of SEQ ID NO: 4, respectively, more preferably at least 96% identity, such as 97% identity, and most preferably 98% identity, such as 99% identity to the amino acid sequence shown in amino acids 19 to 231 of SEQ ID NO: 2 and amino acids 19 to 468 of SEQ ID NO: 4, respectively. Alternatively or additionally, the human or humanized IgG₄ may (only) differ from natalizumab by posttranslational modifications, e.g. by glycosylation. Suitable procedures for changing a glycosylation pattern, such as introducing or deleting a glycosylation site, and assays for determining a glycosylation pattern are well known in the art, and described, for example, in EP 16460039, in particular on page 5 and Examples 4 and 6 therein.

The human or humanized IgG₄ may be recombinantly produced, preferably by using a mammalian cell based expression system. Preferably, said mammalian cell-based expression system is at least one selected from the group consisting of CHO cells (e.g., CHO-K1, CHO-DG44, CHO-DXB), NS/0 cells; BHK cells (e.g., BHK21); SP2/0 cells; HEK cells (e.g., HEK-293); PER-C6 cells, and/or CAP cells.

According to the Scientific Discussion available from the EMEA, natalizumab is recombinantly produced in a NS/0 murine myeloma cell line. The antibody is then purified using Protein A affinity chromatography and hydrophobic interaction chromatography, followed by a buffer exchange and concentration by ultrafiltration/diafiltration. Methods for high density cell cultures of NS/0 cells for, inter alia, producing natalizumab are disclosed in WO 2013/006461, which is incorporated herewith by reference in its entirety. A preferred method for producing natalizumab is disclosed in EP 16460039, in particular Examples 1 and 2 therein, which report a method of culturing CHO DG44 cells under serum-free or protein-free culture conditions, and which express a biosimilar antibody for the monoclonal antibody natalizumab.

The pharmaceutical composition (i)-(iii) of the present disclosure comprise 0.001-0.5% (w/v) of a surfactant. In a preferred embodiment, said formulation comprises 0.005-0.1% (w/v), preferably 0.01-0.05% (w/v), and more preferably 0.02% (w/v) surfactant. Particularly suitable surfactant is a nonionic surfactant, included in the above mentioned concentration ranges. Nonionic surfactants which are pharmaceutically acceptable are known in the art and include polysorbate (Tween), poloxamer (Pluronic), polyethylene glycol alkyl ether (Brij), polyethylene glycol octylphenyl ether (Triton X-100), polypropylene glycol alkyl ether, glucoside alkyl ether, glycerol alkyl ester, and dodecyldimethylamine oxide. Among these polysorbate and poloxamer are particularly preferred, such as polysorbate 80, polysorbate 20, or poloxamer 188, included in the above mentioned concentration ranges. In a most preferred embodiment, the nonionic surfactant is polysorbate 80. Hence, in a preferred embodiment, said pharmaceutical compositions (i)-(iii) of the present disclosure comprise polysorbate 80 in a concentration of 0.005-0.1% (w/v), preferably 0.01-0.05% (w/v), and more preferably 0.02% (w/v).

Formulation (i) comprises, preferably consists of, 1-50 mg/ml (preferably 5-50 mg/ml, more preferably 10-45 mg/ml, even more preferably 15-40 mg/ml, still more preferably 20-30 mg/ml, and most preferably 20 mg/ml) of a human or humanized full-length IgG₄ as further disclosed above, and 10-25 mM histidine buffer, 130-180 mM sodium chloride, and 0.001-0.5% (w/v) of a surfactant as further disclosed above, pH 5.1-6.5. In some embodiments, formulation (i) comprises 10-20 mM histidine buffer; preferably 10-15 mM histidine buffer, more preferably 10-12 mM histidine buffer, and most preferably 10 mM histidine buffer. In principle, any pharmaceutically acceptable histidine buffer may be used, and the histidine may be L-histidine, D-histidine or a mixture of D- and L-histidine. However, in a preferred embodiment the histidine is L-histidine, as this is considered to be most natural and possibly least immunogenic. It further embodiments, formulation (i) comprises 140-170 mM sodium chloride, preferably 145-165 mM sodium chloride, more preferably 150-160 mM sodium chloride, and most preferably 150 mM sodium chloride. The pH of formulation (i) is generally in the range of 5.1-6.5, while preferred embodiments are wherein in formulation (i) pH is 5.1-6.4, preferably 5.1-6.3, more preferably 5.1-6.2, more preferably 5.1-6.1, more preferably 5.2-6.0, more preferably pH is 5.3-5.9, more preferably pH is 5.4-5.8, even more preferably pH is 5.5-5.7. Most preferably formulation (i) has a pH of 5.7.

Particularly preferred formulations (i) are formulations comprising, preferably consisting of

| Formulation | L-histidine | NaCl | polysorbate 80% | |
|---|---|---|---|---|
| # | (mM) | (mM) | (w/v) | pH |
| 1 | 10 | 150 | 0.02 | 5.5 |
| 2 | 12 | 150 | 0.02 | 5.5 |
| 3 | 14 | 150 | 0.02 | 5.5 |
| 4 | 16 | 150 | 0.02 | 5.5 |
| 5 | 18 | 150 | 0.02 | 5.5 |
| 6 | 20 | 150 | 0.02 | 5.5 |
| 7 | 10 | 150 | 0.02 | 5.6 |
| 8 | 12 | 150 | 0.02 | 5.6 |
| 9 | 14 | 150 | 0.02 | 5.6 |
| 10 | 16 | 150 | 0.02 | 5.6 |
| 11 | 18 | 150 | 0.02 | 5.6 |
| 12 | 20 | 150 | 0.02 | 5.6 |
| 13 | 10 | 150 | 0.02 | 5.7 |
| 14 | 12 | 150 | 0.02 | 5.7 |
| 15 | 14 | 150 | 0.02 | 5.7 |
| 16 | 16 | 150 | 0.02 | 5.7 |
| 17 | 18 | 150 | 0.02 | 5.7 |
| 18 | 20 | 150 | 0.02 | 5.7 |
| 19 | 10 | 160 | 0.02 | 5.5 |
| 20 | 12 | 160 | 0.02 | 5.5 |
| 21 | 14 | 160 | 0.02 | 5.5 |
| 22 | 16 | 160 | 0.02 | 5.5 |
| 23 | 18 | 160 | 0.02 | 5.5 |
| 24 | 20 | 160 | 0.02 | 5.5 |
| 25 | 10 | 160 | 0.02 | 5.6 |
| 26 | 12 | 160 | 0.02 | 5.6 |
| 27 | 14 | 160 | 0.02 | 5.6 |
| 28 | 16 | 160 | 0.02 | 5.6 |
| 29 | 18 | 160 | 0.02 | 5.6 |
| 30 | 20 | 160 | 0.02 | 5.6 |
| 31 | 10 | 160 | 0.02 | 5.7 |
| 32 | 12 | 160 | 0.02 | 5.7 |
| 33 | 14 | 160 | 0.02 | 5.7 |
| 34 | 16 | 160 | 0.02 | 5.7 |
| 35 | 18 | 160 | 0.02 | 5.7 |
| 36 | 20 | 160 | 0.02 | 5.7 |
| 37 | 10 | 140 | 0.02 | 5.5 |
| 38 | 12 | 140 | 0.02 | 5.5 |
| 39 | 14 | 140 | 0.02 | 5.5 |
| 40 | 16 | 140 | 0.02 | 5.5 |
| 41 | 18 | 140 | 0.02 | 5.5 |
| 42 | 20 | 140 | 0.02 | 5.5 |
| 43 | 10 | 140 | 0.02 | 5.6 |
| 44 | 12 | 140 | 0.02 | 5.6 |
| 45 | 14 | 140 | 0.02 | 5.6 |
| 46 | 16 | 140 | 0.02 | 5.6 |
| 47 | 18 | 140 | 0.02 | 5.6 |
| 48 | 20 | 140 | 0.02 | 5.6 |
| 49 | 10 | 140 | 0.02 | 5.7 |
| 50 | 12 | 140 | 0.02 | 5.7 |
| 51 | 14 | 140 | 0.02 | 5.7 |
| 52 | 16 | 140 | 0.02 | 5.7 |
| 53 | 18 | 140 | 0.02 | 5.7 |
| 54 | 20 | 140 | 0.02 | 5.7 |

In some embodiments, formulation (i) further comprises 5-30 mM methionine, preferably 6-25 mM methionine, more preferably 7-20 mM methionine, more preferably 8-15 mM methionine, more preferably 9-12 mM methionine, and most preferably 10 mM methionine. In principle, any pharmaceutically acceptable methionine may be used, and the methionine may be L-methionine, D-methionine or a mixture of D- and L-methionine. However, in a preferred embodiment the methionine is L-methionine, as this is considered to be most natural and possibly least immunogenic.

In addition to methionine, formulation (i) may additionally comprises 5-50 mM glycine, preferably 10-45 mM glycine, more preferably 15-40 mM glycine, more preferably 20-35 mM glycine, more preferably 25-30 mM glycine, and most preferably 30 mM glycine.

As shown in the examples, in certain embodiments it can be advantageous to add very low amounts of a phosphate buffer in addition to the histidine buffer. Hence, in some embodiments, formulation (i) further comprises 1-5 mM of a phosphate buffer, preferably 2-4 mM of a phosphate buffer, more preferably 2.5-3 mM of a phosphate buffer, and most preferably 2.5 mM of a phosphate buffer. While in principle any pharmaceutically acceptable phosphate buffer may be used, it is preferred that the phosphate buffer of formulation (i) of the present disclosure is sodium phosphate buffer, e.g., in order to avoid administration of potassium ions which can affect heart function.

Very preferred embodiments of formulation (i) of the present disclosure consists of
10 mM L-histidine buffer, 150 mM NaCl, and 0.02% (w/v) polysorbate 80, pH 5.7.
10 mM L-histidine buffer, 2.5 mM sodium phosphate, 150 mM NaCl, and 0.02% (w/v) polysorbate 80, pH 5.7.
25 mM L-histidine buffer, 150 mM NaCl, and 0.02% (w/v) polysorbate 80, pH 5.7.
10 mM L-histidine buffer, 150 mM NaCl, 10 mM L-methionine, and 0.02% (w/v) polysorbate 80, pH 5.7.
10 mM L-histidine buffer, 2.5 mM sodium phosphate, 150 mM NaCl, 10 mM L-methionine, and 0.02% (w/v) polysorbate 80, pH 5.7.
25 mM L-histidine buffer, 150 mM NaCl, 10 mM L-methionine, and 0.02% (w/v) polysorbate 80, pH 5.7.
10 mM L-histidine buffer, 150 mM NaCl, 10 mM L-methionine, 30 mM glycine, and 0.02% (w/v) polysorbate 80, pH 6.1.
10 mM L-histidine buffer, 2.5 mM sodium phosphate, 150 mM NaCl, 10 mM L-methionine, 30 mM glycine, and 0.02% (w/v) polysorbate 80, pH 6.1.
25 mM L-histidine buffer, 150 mM NaCl, 10 mM L-methionine, 30 mM glycine, and 0.02% (w/v) polysorbate 80, pH 6.1.
10 mM L-histidine buffer, 150 mM NaCl, and 0.2% (w/v) polysorbate 80, pH 5.7.

In a most preferred embodiment, said formulation (i) consists of 10 mM L-histidine buffer, 150 mM NaCl, and 0.02% (w/v) polysorbate 80, pH 5.7.

Formulation (ii) comprises, preferably consists of, 1-50 mg/ml (preferably 5-50 mg/ml, more preferably 10-45 mg/ml, even more preferably 15-40 mg/ml, still more preferably 20-30 mg/ml, and most preferably 20 mg/ml) of a human or humanized full-length IgG₄ as further disclosed above, and 25-30 mM phosphate buffer, 5-10% (w/v) of a polyol (preferably 5% (w/v)), and 0.001-0.5% (w/v) of a surfactant as further disclosed above, pH 5.0-6.9. While in principle any pharmaceutically acceptable phosphate buffer may be used, it is preferred that the phosphate buffer of the formulation (ii) of the present disclosure is sodium phosphate buffer, e.g., in order to avoid administration of potassium ions which can affect heart function. Likewise, in principle any pharmaceutically acceptable polyol may be used, but it is preferred that the polyol in formulation (ii) is selected from sorbitol, mannitol, xylitol, and inositol, among which sorbitol is most preferred. It is further preferred that the pH of formulation (i) is 5.5-6.8, preferably 5.8-6.7, more preferably 6.0-6.5, and most preferably a pH of 6.1-6.3.

Particularly preferred formulations (ii) are

| Formulation | NaPO₄ | sorbitol % | polysorbate 80% | |
|---|---|---|---|---|
| # | (mM) | (w/v) | (w/v) | pH |
| 55 | 25 | 5 | 0.02 | 6.0 |
| 56 | 26 | 5 | 0.02 | 6.0 |
| 57 | 27 | 5 | 0.02 | 6.0 |
| 58 | 28 | 5 | 0.02 | 6.0 |
| 59 | 29 | 5 | 0.02 | 6.0 |
| 60 | 30 | 5 | 0.02 | 6.0 |
| 61 | 25 | 5 | 0.02 | 6.1 |
| 62 | 26 | 5 | 0.02 | 6.1 |
| 63 | 27 | 5 | 0.02 | 6.1 |
| 64 | 28 | 5 | 0.02 | 6.1 |
| 65 | 29 | 5 | 0.02 | 6.1 |
| 66 | 30 | 5 | 0.02 | 6.1 |
| 67 | 25 | 5 | 0.02 | 6.2 |
| 68 | 26 | 5 | 0.02 | 6.2 |
| 69 | 27 | 5 | 0.02 | 6.2 |
| 70 | 28 | 5 | 0.02 | 6.2 |
| 71 | 29 | 5 | 0.02 | 6.2 |
| 72 | 30 | 5 | 0.02 | 6.2 |
| 73 | 25 | 5 | 0.02 | 6.3 |
| 74 | 26 | 5 | 0.02 | 6.3 |
| 75 | 27 | 5 | 0.02 | 6.3 |
| 76 | 28 | 5 | 0.02 | 6.3 |
| 77 | 29 | 5 | 0.02 | 6.3 |
| 78 | 30 | 5 | 0.02 | 6.3 |
| 79 | 25 | 7 | 0.02 | 6.0 |
| 80 | 26 | 7 | 0.02 | 6.0 |
| 81 | 27 | 7 | 0.02 | 6.0 |
| 82 | 28 | 7 | 0.02 | 6.0 |
| 83 | 29 | 7 | 0.02 | 6.0 |
| 84 | 30 | 7 | 0.02 | 6.0 |
| 85 | 25 | 7 | 0.02 | 6.1 |
| 86 | 26 | 7 | 0.02 | 6.1 |
| 87 | 27 | 7 | 0.02 | 6.1 |
| 88 | 28 | 7 | 0.02 | 6.1 |
| 89 | 29 | 7 | 0.02 | 6.1 |
| 90 | 30 | 7 | 0.02 | 6.1 |
| 91 | 25 | 7 | 0.02 | 6.2 |
| 92 | 26 | 7 | 0.02 | 6.2 |
| 93 | 27 | 7 | 0.02 | 6.2 |
| 94 | 28 | 7 | 0.02 | 6.2 |
| 95 | 29 | 7 | 0.02 | 6.2 |
| 96 | 30 | 7 | 0.02 | 6.2 |
| 97 | 25 | 7 | 0.02 | 6.3 |
| 98 | 26 | 7 | 0.02 | 6.3 |
| 99 | 27 | 7 | 0.02 | 6.3 |
| 100 | 28 | 7 | 0.02 | 6.3 |
| 101 | 29 | 7 | 0.02 | 6.3 |
| 102 | 30 | 7 | 0.02 | 6.3 |
| 103 | 25 | 10 | 0.02 | 6.0 |
| 104 | 26 | 10 | 0.02 | 6.0 |
| 105 | 27 | 10 | 0.02 | 6.0 |
| 106 | 28 | 10 | 0.02 | 6.0 |
| 107 | 29 | 10 | 0.02 | 6.0 |
| 108 | 30 | 10 | 0.02 | 6.0 |
| 109 | 25 | 10 | 0.02 | 6.1 |
| 110 | 26 | 10 | 0.02 | 6.1 |
| 111 | 27 | 10 | 0.02 | 6.1 |
| 112 | 28 | 10 | 0.02 | 6.1 |
| 113 | 29 | 10 | 0.02 | 6.1 |
| 114 | 30 | 10 | 0.02 | 6.1 |
| 115 | 25 | 10 | 0.02 | 6.2 |
| 116 | 26 | 10 | 0.02 | 6.2 |
| 117 | 27 | 10 | 0.02 | 6.2 |
| 118 | 28 | 10 | 0.02 | 6.2 |
| 119 | 29 | 10 | 0.02 | 6.2 |
| 120 | 30 | 10 | 0.02 | 6.2 |
| 121 | 25 | 10 | 0.02 | 6.3 |
| 122 | 26 | 10 | 0.02 | 6.3 |
| 123 | 27 | 10 | 0.02 | 6.3 |
| 124 | 28 | 10 | 0.02 | 6.3 |
| 125 | 29 | 10 | 0.02 | 6.3 |
| 126 | 30 | 10 | 0.02 | 6.3 |

In some embodiments, formulation (ii) further comprises 5-30 mM arginine-HCl, preferably 6-25 mM arginine-HCl, more preferably 7-20 mM arginine-HCl, more preferably 8-15 mM arginine-HCl, more preferably 9-12 mM arginine-HCl, and most preferably 10 mM arginine-HCl. In principle, any pharmaceutically acceptable arginine-HCl may be used, and the arginine-HCl may be L-arginine-HCl, D-arginine-HCl or a mixture of D- and L-arginine-HCl. However, in a preferred embodiment the arginine-HCl is L-arginine-HCl, as this is considered to be most natural and possibly least immunogenic.

In addition to arginine-HCl, formulation (ii) may additionally comprise 0.5-10 mM methionine, preferably 0.75-5 mM methionine, more preferably 1-3 mM methionine, more preferably 1.5-2.5 mM methionine, and most preferably 2 mM methionine. In principle, any pharmaceutically acceptable methionine may be used, and the methionine may be L-methionine, D-methionine or a mixture of D- and L-methionine. However, in a preferred embodiment the methionine is L-methionine, as this is considered to be most natural and possibly least immunogenic.

In still further embodiments, formulation (ii) may further comprise 10-100 mM NaCl, preferably 20-80 mM NaCl, more preferably 25-75 mM NaCl, more preferably 30-70 mM NaCl, more preferably 40-60 mM NaCl, more preferably 45-55 mM NaCl, and most preferably 50 mM NaCl.

Very preferred embodiments of formulation (ii) of the present disclosure consists of
25-30 mM sodium phosphate buffer, 5% (w/v) sorbitol, and 0.02% (w/v) polysorbate 80, pH 6.5.
25-30 mM sodium phosphate buffer, 5% (w/v) sorbitol, 50 mM NaCl, and 0.02% (w/v) polysorbate 80, pH 5.7-6.5.
25-30 mM sodium phosphate buffer, 5% (w/v) sorbitol, 50 mM NaCl, and 0.02% (w/v) polysorbate 80, pH 5.7.
25-30 mM sodium phosphate buffer, 5% (w/v) sorbitol, 10 mM L-arginine-HCl, and 0.02% (w/v) polysorbate 80, pH 6.1.
25-30 mM sodium phosphate buffer, 5% (w/v) sorbitol, 10 mM L-arginine-HCl, 2 mM L-methionine, and 0.02% (w/v) polysorbate 80, pH 6.1.
30 mM sodium phosphate buffer, 5% (w/v) sorbitol, and 0.02% (w/v) polysorbate 80, pH 6.5.
30 mM sodium phosphate buffer, 5% (w/v) sorbitol, 50 mM NaCl, and 0.02% (w/v) polysorbate 80, pH 5.7-6.5.
30 mM sodium phosphate buffer, 5% (w/v) sorbitol, 50 mM NaCl, and 0.02% (w/v) polysorbate 80, pH 5.7.
30 mM sodium phosphate buffer, 5% (w/v) sorbitol, 10 mM L-arginine-HCl, and 0.02% (w/v) polysorbate 80, pH 6.1.
30 mM sodium phosphate buffer, 5% (w/v) sorbitol, 10 mM L-arginine-HCl, 2 mM L-methionine, and 0.02% (w/v) polysorbate 80, pH 6.1.

In a most preferred embodiment, said formulation (i) is 25-30 mM (most preferably 30 mM) sodium phosphate buffer, 5% (w/v) sorbitol, and 0.02% (w/v) polysorbate 80, pH 6.5.

Formulation (iii) comprises, preferably consists of, 1-50 mg/ml (preferably 5-50 mg/ml, more preferably 10-45 mg/ml, even more preferably 15-40 mg/ml, still more preferably 20-30 mg/ml, and most preferably 20 mg/ml) of a human or humanized full-length IgG₄ as further disclosed above, and 10-25 mM histidine buffer, 10-50 mM sodium chloride, and 0.001-0.5% (w/v) of a surfactant of a surfactant as further disclosed above, pH 5.1-6.5. In some embodiments, formulation (iii) comprises 10-20 mM histidine buffer; preferably 10-15 mM histidine buffer, more preferably 10-12 mM histidine buffer, and most preferably 10 mM histidine buffer. In principle, any pharmaceutically acceptable histidine buffer may be used, and the histidine may be L-histidine, D-histidine or a mixture of D- and L-histidine. However, in a preferred embodiment the histidine is L-histidine, as this is considered to be most natural and possibly least immunogenic. It further embodiments, formulation (iii) comprises 15-40 mM sodium chloride, preferably 20-30 mM sodium chloride, more preferably 20-25 mM sodium chloride, and most preferably 20 mM sodium chloride. The pH of formulation (iii) is generally in the range of 5.1-6.5, while preferred embodiments are wherein in formulation (iii) pH is 5.1-6.4, preferably 5.1-6.3, more preferably 5.1-6.2, more preferably 5.1-6.1, more preferably 5.2-6.0, more preferably pH is 5.3-5.9, more preferably pH is 5.4-5.8, even more preferably pH is 5.5-5.7. Most preferably formulation (iii) has a pH of 5.7.

As shown in the examples, in certain embodiments it can be advantageous to add very low amounts of a phosphate buffer in addition to the histidine buffer. Hence, in some embodiments, formulation (iii) further comprises 1-5 mM of a phosphate buffer, preferably 2-4 mM of a phosphate buffer, more preferably 2.5-3 mM of a phosphate buffer, and most preferably 2.5 mM of a phosphate buffer. While in principle any pharmaceutically acceptable phosphate buffer may be used, it is preferred that the phosphate buffer of formulation (iii) of the present disclosure is sodium phosphate buffer, e.g., in order to avoid administration of potassium ions which can affect heart function.

In some embodiments, formulation (iii) further comprises 5-30 mM methionine, preferably 6-25 mM methionine, more preferably 7-20 mM methionine, more preferably 8-15 mM methionine, more preferably 9-12 mM methionine, and most preferably 10 mM methionine. In principle, any pharmaceutically acceptable methionine may be used, and the methionine may be L-methionine, D-methionine or a mixture of D- and L-methionine. However, in a preferred embodiment the methionine is L-methionine, as this is considered to be most natural and possibly least immunogenic.

In addition to methionine, formulation (iii) may additionally comprises 5-50 mM glycine, preferably 10-45 mM glycine, more preferably 15-40 mM glycine, more preferably 20-35 mM glycine, more preferably 25-30 mM glycine, and most preferably 30 mM glycine.

In still further embodiments, formulation (iii) may further comprise 5-10% (w/v) of a polyol, preferably 5-9% (w/v) of a polyol, more preferably 5-8% (w/v) of a polyol, more preferably 5-7% (w/v) of a polyol, more preferably 5-6% (w/v) of a polyol, and most preferably 5% (w/v) of a polyol. In principle, any pharmaceutically acceptable polyol may be used. However, in preferred embodiments, the polyol is selected from sorbitol, sucrose, mannitol, xylitol, and inositol; more preferably wherein the polyol is sorbitol or sucrose, and most preferably the polyol is sorbitol. Accordingly, in one preferred embodiments, formulation (iii) further comprises 5% (w/v) of sorbitol.

Very preferred embodiments of formulation (iii) of the present disclosure consists of
10 mM L-histidine buffer, 20 mM NaCl, and 0.02% (w/v) polysorbate 80, pH 5.7.
10 mM L-histidine buffer, 20 mM NaCl, 5% (w/v) of sorbitol, and 0.02% (w/v) polysorbate 80, pH 5.7.
10 mM L-histidine buffer, 2.5 mM sodium phosphate, 20 mM NaCl, and 0.02% (w/v) polysorbate 80, pH 5.7.
10 mM L-histidine buffer, 2.5 mM sodium phosphate, 20 mM NaCl, 10 mM L-methionine, and 0.02% (w/v) polysorbate 80, pH 5.7.
10 mM L-histidine buffer, 2.5 mM sodium phosphate, 20 mM NaCl, 10 mM L-methionine, 30 mM glycine, and 0.02% (w/v) polysorbate 80, pH 6.1.

The conductivity of a formulation correlates with the presence of ions in the formulation. A higher conductivity can be advantageous depending on the molecules and its surface charge distribution. In the present case, without wishing to be bound by theory, it is assumed that a higher conductivity will break long range surface charge-charge interactions in the human or humanized IgG₄. These long range surface charge-charge interactions in proteins are known to lead to a higher aggregation. Therefore, in a preferred embodiment, the conductivity of the pharmaceutical composition of the present disclosure is at least 2.5 mS/cm, preferably at least 2.6 mS/cm. Consequently, in formulations like formulation (ii) described herein which do not contain salts such as sodium chloride, stability of the human or humanized IgG₄ is increased by increasing the buffer concentration. On the other hand, the presence of too many charged ions in the solution may lead to charge screening and a salting-out effect, which shall be avoided. Consequently, in formulations like formulation (i) which contain sodium chloride, stability of human or humanized IgG₄ is improved, since the buffer concentration is rather kept low. It is believed that within the indicated concentration ranges for the pharmaceutical compositions no salting-out effects are observed, for which reasons it is not necessary to indicate an upper limit for the conductivity.

Accordingly, in view of the foregoing, the present disclosure also provides a method for stabilizing a human or humanized full length IgG₄, comprising combining 1-50 mg/ml (preferably 5-50 mg/ml, more preferably 10-45 mg/ml, even more preferably 15-40 mg/ml, still more preferably 20-30 mg/ml, and most preferably 20 mg/ml) of a human or humanized full-length IgG₄ with a formulation selected from
(i) a formulation comprising 10-25 mM histidine buffer, 130-180 mM sodium chloride, and 0.001-0.5% (w/v) of a surfactant, pH 5.1-6.5; or
(ii) a formulation comprising 25-30 mM phosphate buffer, 5-10% (w/v) of a polyol, and 0.001-0.5% (w/v) of a surfactant, pH 5.0-6.9; or
(iii) a formulation comprising 10-25 mM histidine buffer, 10-50 mM sodium chloride, and 0.001-0.5% (w/v) of a surfactant, pH 5.1-6.5;
   thereby preparing a stable aqueous pharmaceutical composition (i)-(iii) as described above. Preferred embodiments of the formulations (i)-(iii) added to the human or humanized full-length IgG₄ are as further disclosed for formulations (i)-(iii) above. As also noted above, it is preferred that the IgG₄ is a humanized IgG₄, in particular wherein the human or humanized full length IgG₄ is essentially identical/similar to natalizumab, more preferably wherein the IgG₄ is natalizumab, as further disclosed in detail above. In addition, it is also preferred that the final aqueous pharmaceutical composition has a conductivity of at least 2.5 mS/cm, preferably at least 2.6 mS/cm for the above indicated reasons.

Likewise, the present disclosure also provides a method for producing an aqueous pharmaceutical composition (i)-(iii) as described above, comprising combining 1-50 mg/ml (preferably 5-50 mg/ml, more preferably 10-45 mg/ml, even more preferably 15-40 mg/ml, still more preferably 20-30 mg/ml, and most preferably 20 mg/ml) of a human or humanized full length IgG₄ with a formulation selected from
(i) a formulation comprising 10-25 mM histidine buffer, 130-180 mM sodium chloride, and 0.001-0.5% (w/v) of a surfactant, pH 5.1-6.5; or
(ii) a formulation comprising 25-30 mM phosphate buffer, 5-10% (w/v) of a polyol, and 0.001-0.5% (w/v) of a surfactant, pH 5.0-6.9; or
(iii) a formulation comprising 10-25 mM histidine buffer, 10-50 mM sodium chloride, and 0.001-0.5% (w/v) of a surfactant, pH 5.1-6.5.

Preferred embodiments of the formulation (i)-(iii) added to the human or humanized full-length IgG₄ are as further disclosed above. As also noted above, it is preferred that the IgG₄ is a humanized IgG₄, in particular wherein the human or humanized full length IgG₄ is essentially identical/similar to natalizumab, more preferably wherein the IgG₄ is natalizumab, as further disclosed in detail above. In addition, it is also preferred that the final aqueous pharmaceutical composition has a conductivity of at least 2.5 mS/cm, preferably at least 2.6 mS/cm for the above indicated reasons.

The present disclosure further provides an article of manufacture, comprising a pharmaceutical composition of the present disclosure and instructions for use of said composition. In preferred embodiments, said pharmaceutical composition is contained in a pre-filled vial or in a pre-filled syringe, wherein it is more preferred that said pharmaceutical composition is contained in a pre-filled vial. Suitable vials and syringes for pharmaceutical purposes, e.g., those described in the examples section below, are known and used in the field, and can be obtained from commercial suppliers.

Since the pharmaceutical compositions are intended for in vivo administration, in particular by infusion, it is clear to the skilled person that the final pharmaceutical compositions are required to be sterile. The pharmaceutical composition may be administered at a suitable dose, i.e. about 1 ng/kg body weight to about 100 mg/kg body weight of a subject, preferably at a dose of about 10 ng/kg to about 10 mg/kg, more preferably at a dose of about 10 ng/kg to about 5 mg/kg per body weight. However, the dosage regimen will be determined by an attending physician and depend upon many factors, including the therapeutic IgG₄ to be administered, patient's size and condition, body surface area, age, sex, time and route of administration, and on other drugs being administered concurrently. Administration will preferably be intravenously but may also be subcutaneously, intramuscularly, or intraperitoneally.

Natalizumab is marketed by Biogen MA, Inc. under the name Tysabri, and has FDA-approval for the treatment of multiple sclerosis and Crohn's disease, and EMEA approval for the treatment of multiple sclerosis. Recently, it was suggested that natalizumab could also be used in a combination treatment of B-cell malignancies, where it is intended to overcome the resistance to rituximab. Accordingly, it is to be expected that the pharmaceutical composition of the present disclosure can be suitably applied in the treatment of multiple sclerosis or Crohn's disease, and possibly in the treatment of B-cell malignancies. Administration of natalizumab may be carried out as follows: 15 mL concentrate from the vial will be extracted and diluted in 100 mL 0.9% NaCl. The bag will be gently inverted, but not agitated. After dilution, the solution will be used immediately or within 8 hr (keep refrigerated at 2-8° C., do not freeze it). The infusion of equilibrated to room temperature solution will be done in 1 hour, followed by a flash with 0.9% NaCl.

The invention is further described by the following embodiments.
1. An aqueous pharmaceutical composition comprising 1-50 mg/ml of a human or humanized full-length IgG₄ in a formulation selected from
   (i) a formulation comprising 10-25 mM histidine buffer, 130-180 mM sodium chloride, and 0.001-0.5% (w/v) of a surfactant, pH 5.1-6.5; or
   (ii) a formulation comprising 25-30 mM phosphate buffer, 5-10% (w/v) of a polyol, and 0.001-0.5% (w/v) of a surfactant, pH 5.0-6.9; or
   (iii) a formulation comprising 10-25 mM histidine buffer, 10-50 mM sodium chloride, and 0.001-0.5% (w/v) of a surfactant, pH 5.1-6.5.
2. The pharmaceutical composition of embodiment 1, wherein said pharmaceutical composition comprises 5-50 mg/ml, preferably 10-45 mg/ml, more preferably 15-40 mg/ml, even more preferably 20-30 mg/ml of said human or humanized full-length IgG₄.
3. The pharmaceutical composition of embodiment 1 or 2, wherein the IgG₄ is a humanized IgG₄.
4. The pharmaceutical composition of any one of embodiments 1-3, wherein the human or humanized full-length IgG₄ is natalizumab.
5. The pharmaceutical composition of any one of embodiments 1-4, wherein said formulation comprises 0.005-0.1% (w/v), preferably 0.01-0.05% (w/v), and more preferably 0.02% (w/v) surfactant.
6. The pharmaceutical composition of any one of embodiments 1-5, wherein the surfactant is a nonionic surfactant.
7. The pharmaceutical composition of embodiment 6, wherein the nonionic surfactant is selected from polysorbate (Tween), poloxamer (Pluronic), polyethylene glycol alkyl ether (Brij), polyethylene glycol octylphenyl ether (Triton X-100), polypropylene glycol alkyl ether, glucoside alkyl ether, glycerol alkyl ester, and dodecyldimethylamine oxide; preferably wherein the nonionic surfactant is polysorbate or poloxamer; more preferably wherein the nonionic surfactant is polysorbate 80, polysorbate 20, or poloxamer 188.
8. The pharmaceutical composition of embodiment 6, wherein the nonionic surfactant is polysorbate, preferably, wherein the nonionic surfactant is polysorbate 80.
9. The pharmaceutical composition of any one of embodiments 1-8, wherein formulation (i) comprises 10-20 mM histidine buffer; preferably 10-15 mM histidine buffer, more preferably 10-12 mM histidine buffer, and most preferably 10 mM histidine buffer.
10. The pharmaceutical composition of any one of embodiments 1-9, wherein said histidine in formulation (i) is L-histidine.
11. The pharmaceutical composition of any one of embodiments 1-10, wherein formulation (i) comprises 140-170 mM sodium chloride, preferably 145-165 mM sodium chloride, more preferably 150-160 mM sodium chloride, and most preferably 150 mM sodium chloride.
12. The pharmaceutical composition of any one of embodiments 1-11, wherein in formulation (i) the pH is 5.1-6.4, preferably pH is 5.1-6.3, more preferably the pH is 5.1-6.2, more preferably the pH is 5.1-6.1, more preferably pH is 5.2-6.0, more preferably pH is 5.3-5.9, more preferably pH is 5.4-5.8, even more preferably pH is 5.5-5.7, and most preferably formulation (i) has a pH of 5.7.
13. The pharmaceutical composition of any one of embodiments 1-12, wherein formulation (i) further comprises 5-30 mM methionine, preferably 6-25 mM methionine, more preferably 7-20 mM methionine, more preferably 8-15 mM methionine, more preferably 9-12 mM methionine, and most preferably 10 mM methionine.
14. The pharmaceutical composition of embodiment 13, wherein the methionine is L-methionine.
15. The pharmaceutical composition of any one of embodiments 13 or 14, wherein formulation (i) further comprises 5-50 mM glycine, preferably 10-45 mM glycine, more preferably 15-40 mM glycine, more preferably 20-35 mM glycine, more preferably 25-30 mM glycine, and most preferably 30 mM glycine.
16. The pharmaceutical composition of any one of embodiments 1-15, wherein formulation (i) further comprises 1-5 mM of a phosphate buffer, preferably 2-4 mM of a phosphate buffer, more preferably 2.5-3 mM of a phosphate buffer, and most preferably 2.5 mM of a phosphate buffer.
17. The pharmaceutical composition of embodiment 16, wherein said phosphate buffer is sodium phosphate buffer.
18. The pharmaceutical composition of any one of embodiments 1-4, wherein said formulation (i) consists of 10 mM L-histidine buffer, 150 mM NaCl, and 0.02% (w/v) polysorbate 80, pH 5.7.
19. The pharmaceutical composition of any one of embodiments 1-4, wherein said formulation (i) consists of 10 mM L-histidine buffer, 2.5 mM sodium phosphate, 150 mM NaCl, and 0.02% (w/v) polysorbate 80, pH 5.7.
20. The pharmaceutical composition of any one of embodiments 1-4, wherein said formulation (i) consists of 25 mM L-histidine buffer, 150 mM NaCl, and 0.02% (w/v) polysorbate 80, pH 5.7.
21. The pharmaceutical composition of any one of embodiments 1-4, wherein said formulation (i) consists of 10 mM L-histidine buffer, 150 mM NaCl, 10 mM L-methionine, and 0.02% (w/v) polysorbate 80, pH 5.7.
22. The pharmaceutical composition of any one of embodiments 1-4, wherein said formulation (i) consists of 10 mM L-histidine buffer, 2.5 mM sodium phosphate, 150 mM NaCl, 10 mM L-methionine, and 0.02% (w/v) polysorbate 80, pH 5.7.
23. The pharmaceutical composition of any one of embodiments 1-4, wherein said formulation (i) consists of 25 mM L-histidine buffer, 150 mM NaCl, 10 mM L-methionine, and 0.02% (w/v) polysorbate 80, pH 5.7.
24. The pharmaceutical composition of any one of embodiments 1-4, wherein said formulation (i) consists of 10 mM L-histidine buffer, 150 mM NaCl, 10 mM L-methionine, 30 mM glycine, and 0.02% (w/v) polysorbate 80, pH 6.1.
25. The pharmaceutical composition of any one of embodiments 1-4, wherein said formulation (i) consists of 10 mM L-histidine buffer, 2.5 mM sodium phosphate, 150 mM NaCl, 10 mM L-methionine, 30 mM glycine, and 0.02% (w/v) polysorbate 80, pH 6.1.
26. The pharmaceutical composition of any one of embodiments 1-4, wherein said formulation (i) consists of 25 mM L-histidine buffer, 150 mM NaCl, 10 mM L-methionine, 30 mM glycine, and 0.02% (w/v) polysorbate 80, pH 6.1.
27. The pharmaceutical composition of any one of embodiments 1-4, wherein said formulation (i) consists of 10 mM L-histidine buffer, 150 mM NaCl, and 0.2% (w/v) polysorbate 80, pH 5.7.
28. The pharmaceutical composition of any one of embodiments 1-8, wherein in formulation (ii) said phosphate buffer is sodium phosphate buffer.
29. The pharmaceutical composition of any one of embodiments 1-8 or 28, wherein the polyol in formulation (ii) is selected from sorbitol, sucrose, mannitol, xylitol, and inositol; preferably wherein the polyol is sorbitol or sucrose, more preferably wherein the polyol is sorbitol.
30. The pharmaceutical composition of any one of embodiments 1-8 or 28-29, wherein formulation (ii) comprises 5% (w/v) of said polyol.
31. The pharmaceutical composition of any one of embodiments 1-8 or 28-30, wherein in formulation (ii) pH is 5.5-6.8, preferably 5.7-6.7, more preferably 5.8-6.7, more preferably 5.8-6.5, more preferably 6.0-6.5, and most preferably a pH of 6.1-6.3.
32. The pharmaceutical composition of any one of embodiments 1-8 or 28-31, wherein formulation (ii) further comprises 5-30 mM arginine-HCl, preferably 6-25 mM arginine-HCl, more preferably 7-20 mM arginine-HCl, more preferably 8-15 mM arginine-HCl, more preferably 9-12 mM arginine-HCl, and most preferably 10 mM arginine-HCl.
33. The pharmaceutical composition of embodiment 32, wherein the arginine-HCl is L-arginine-HCl.
34. The pharmaceutical composition of any one of embodiments 32 or 33, wherein formulation (ii) further comprises 0.5-10 mM methionine, preferably 0.75-5 mM methionine, more preferably 1-3 mM methionine, more preferably 1.5-2.5 mM methionine, and most preferably 2 mM methionine.
35. The pharmaceutical composition of embodiment 34, wherein the methionine is L-methionine.
36. The pharmaceutical composition of any one of embodiments 1-8 or 28-31, wherein formulation (ii) further comprises 10-100 mM NaCl, preferably 20-80 mM NaCl, more preferably 25-75 mM NaCl, more preferably 30-70 mM NaCl, more preferably 40-60 mM NaCl, more preferably 45-55 mM NaCl, and most preferably 50 mM NaCl.
37. The pharmaceutical composition of any one of embodiments 1-4, wherein said formulation (ii) consists of 25-30 mM sodium phosphate buffer, 5% (w/v) sorbitol, and 0.02% (w/v) polysorbate 80, pH 6.5.
38. The pharmaceutical composition of any one of embodiments 1-4, wherein said formulation (ii) consists of 25-30 mM sodium phosphate buffer, 5% (w/v) sorbitol, 50 mM NaCl, and 0.02% (w/v) polysorbate 80, pH 5.7-6.5.
39. The pharmaceutical composition of embodiment 38, wherein the pH is 5.7.
40. The pharmaceutical composition of any one of embodiments 1-4, wherein said formulation (ii) consists of 25-30 mM sodium phosphate buffer, 5% (w/v) sorbitol, 10 mM L-arginine-HCl, and 0.02% (w/v) polysorbate 80, pH 6.1.
41. The pharmaceutical composition of embodiment 40, further comprising 2 mM L-methionine.
42. The pharmaceutical composition of any one of embodiments 37-40, wherein formulation (ii) comprises 30 mM sodium phosphate.
43. The pharmaceutical composition of any one of embodiments 1-8, wherein formulation (iii) comprises 10-20 mM histidine buffer; preferably 10-15 mM histidine buffer, more preferably 10-12 mM histidine buffer, and most preferably 10 mM histidine buffer.
44. The pharmaceutical composition of any one of embodiments 1-8 or 43, wherein said histidine in formulation (iii) is L-histidine.
45. The pharmaceutical composition of any one of embodiments 1-8 or 43-44, wherein formulation (iii) comprises 15-40 mM sodium chloride, preferably 20-30 mM sodium chloride, more preferably 20-25 mM sodium chloride, and most preferably 20 mM sodium chloride.
46. The pharmaceutical composition of any one of embodiments 1-8 or 43-45, wherein in formulation (iii) the pH is 5.1-6.4, preferably pH is 5.1-6.3, more preferably the pH is 5.1-6.2, more preferably the pH is 5.1-6.1, more preferably pH is 5.2-6.0, more preferably pH is 5.3-5.9, more preferably pH is 5.4-5.8, even more preferably pH is 5.5-5.7, and most preferably formulation (iii) has a pH of 5.7.
47. The pharmaceutical composition of any one of embodiments 1-8 or 43-46, wherein formulation (iii) further comprises 1-5 mM of a phosphate buffer, preferably 2-4 mM of a phosphate buffer, more preferably 2.5-3 mM of a phosphate buffer, and most preferably 2.5 mM of a phosphate buffer.
48. The pharmaceutical composition of embodiment 47, wherein said phosphate buffer is sodium phosphate buffer.
49. The pharmaceutical composition of any one of embodiments 1-8 or 43-48, wherein formulation (iii) further comprises 5-30 mM methionine, preferably 6-25 mM methionine, more preferably 7-20 mM methionine, more preferably 8-15 mM methionine, more preferably 9-12 mM methionine, and most preferably 10 mM methionine.
50. The pharmaceutical composition of embodiment 49, wherein the methionine is L-methionine.
51. The pharmaceutical composition of any one of embodiments 49 or 50, wherein formulation (iii) further comprises 5-50 mM glycine, preferably 10-45 mM glycine, more preferably 15-40 mM glycine, more preferably 20-35 mM glycine, more preferably 25-30 mM glycine, and most preferably 30 mM glycine.
52. The pharmaceutical composition of any one of embodiments 1-8 or 43-46, wherein formulation (iii) further comprises 5-10% (w/v) of a polyol, preferably 5-9% (w/v) of a polyol, more preferably 5-8% (w/v) of a polyol, more preferably 5-7% (w/v) of a polyol, more preferably 5-6% (w/v) of a polyol, and most preferably 5% (w/v) of a polyol.
53. The pharmaceutical composition of embodiment 52, wherein the polyol is selected from sorbitol, sucrose, mannitol, xylitol, and inositol; preferably wherein the polyol is sorbitol or sucrose, more preferably wherein the polyol is sorbitol.
54. The pharmaceutical composition of embodiment 53, wherein formulation (iii) comprises 5% (w/v) of sorbitol.
55. The pharmaceutical composition of any one of embodiments 1-4, wherein said formulation (iii) consists of 10 mM L-histidine buffer, 20 mM NaCl, and 0.02% (w/v) polysorbate 80, pH 5.7.
56. The pharmaceutical composition of any one of embodiments 1-4, wherein said formulation (iii) consists of 10 mM L-histidine buffer, 20 mM NaCl, 5% (w/v) of sorbitol, and 0.02% (w/v) polysorbate 80, pH 5.7.
57. The pharmaceutical composition of any one of embodiments 1-4, wherein said formulation (iii) consists of 10 mM L-histidine buffer, 2.5 mM sodium phosphate, 20 mM NaCl, and 0.02% (w/v) polysorbate 80, pH 5.7.
58. The pharmaceutical composition of any one of embodiments 1-4, wherein said formulation (iii) consists of 10 mM L-histidine buffer, 2.5 mM sodium phosphate, 20 mM NaCl, 10 mM L-methionine, and 0.02% (w/v) polysorbate 80, pH 5.7.
59. The pharmaceutical composition of any one of embodiments 1-4, wherein said formulation (iii) consists of 10 mM L-histidine buffer, 2.5 mM sodium phosphate, 20 mM NaCl, 10 mM L-methionine, 30 mM glycine, and 0.02% (w/v) polysorbate 80, pH 6.1.
60. The pharmaceutical composition of any one of embodiments 1-59, wherein the conductivity of said pharmaceutical composition is at least 2.5 mS/cm, preferably at least 2.6 mS/cm.
61. An article of manufacture, comprising a pharmaceutical composition according to any one of embodiments 1-60 and instructions for use of said composition, preferably wherein said pharmaceutical composition is contained in a pre-filled vial or in a pre-filled syringe, more preferably wherein said pharmaceutical composition is contained in a pre-filled vial.
62. A method for stabilizing a human or humanized full length IgG₄, comprising combining 1-50 mg/ml of a human or humanized full-length IgG₄ with a formulation selected from
   (i) a formulation comprising 10-25 mM histidine buffer, 130-180 mM sodium chloride, and 0.001-0.5% (w/v) of a surfactant, pH 5.1-6.5; or
   (ii) a formulation comprising 25-30 mM phosphate buffer, 5-10% (w/v) of a polyol, and 0.001-0.5% (w/v) of a surfactant, pH 5.0-6.9; or
   (iii) a formulation comprising 10-25 mM histidine buffer, 10-50 mM sodium chloride, and 0.001-0.5% (w/v) of a surfactant, pH 5.1-6.5;
   thereby preparing a stable aqueous pharmaceutical composition.
63. A method for producing an aqueous pharmaceutical composition according to any one of embodiments 1-60, comprising combining 1-50 mg/ml of a human or humanized full length IgG₄ with a formulation selected from
   (i) a formulation comprising 10-25 mM histidine buffer, 130-180 mM sodium chloride, and 0.001-0.5% (w/v) of a surfactant, pH 5.1-6.5; or
   (ii) a formulation comprising 25-30 mM phosphate buffer, 5-10% (w/v) of a polyol, and 0.001-0.5% (w/v) of a surfactant, pH 5.0-6.9; or
   (iii) a formulation comprising 10-25 mM histidine buffer, 10-50 mM sodium chloride, and 0.001-0.5% (w/v) of a surfactant, pH 5.1-6.5.
64. The method of embodiment 62 or embodiment 63, wherein the human or humanized full-length IgG₄ is added in an amount of 5-50 mg/ml, preferably 10-45 mg/ml, more preferably 15-40 mg/ml, even more preferably 20-30 mg/ml of said human or humanized full-length IgG₄.
65. The method of any one of embodiments 62-64, wherein the IgG₄ is a humanized IgG₄.
66. The method of any one of embodiments 62-65, wherein the human or humanized full-length IgG₄ is natalizumab.
67. The method of any one of embodiments 62-66, wherein the formulation added to the human or humanized full-length IgG₄ is as further defined in embodiments 5-59.
68. The method of any one of embodiments 62-67, wherein the final stable aqueous pharmaceutical composition has a conductivity of at least 2.5 mS/cm, preferably at least 2.6 mS/cm.
69. An aqueous pharmaceutical composition according to any one of embodiments 1-60 for use in the treatment of multiple sclerosis or Crohn's disease.

In the following, the present invention as defined in the embodiments is further illustrated by the following examples, which are not intended to limit the scope of the present invention. All references cited herein are explicitly incorporated by reference.

### DESCRIPTION OF THE SEQUENCES

The start and stop codons are indicated in bold, the signal sequence is underlined.

### Natalizumab Light Chain

(SEQ ID NO: 1)
Atgaagtgggtgaccttcatctccctgctgtttctgttctcctccgccta
ctccgacatccagatgacccagtccccctccagcctgtccgcctccgtgg
gcgacagagtgaccatcacatgcaagacctcccaggacatcaacaagtac
atggcctggtatcagcagacccccggcaaggcccctcggctgctgatcca
ctacacctccgccctgcagcccggcatcccttccagattctccggctctg
gctctggccgggactacaccttcaccatctccagcctgcagcctgaggac
attgccacctactactgcctgcagtacgacaacctgtggaccttcggcca
gggcaccaaggtggaaatcaagcggaccgtggccgctccctccgtgttca
tcttcccaccctccgacgagcagctgaagtccggcaccgccagcgtggtg
tgcctgctgaacaacttctacccccgcgaggccaaggtgcagtggaaggt
ggacaacgccctgcagagcggcaactcccaggaatccgtgaccgagcagg
actccaaggacagcacctactccctgtcctccaccctgaccctgtccaag
gccgactacgagaagcacaaggtgtacgcctgcgaagtgacccaccaggg
cctgtccagccccgtgaccaagtccttcaaccggggcgagtgctgatag

### Translation

(SEQ ID NO: 2)
MKWVTFISLLFLFSSAYSDIQMTQSPSSLSASVGDRVTITCKTSQDINKY
MAWYQQTPGKAPRLLIHYTSALQPGIPSRFSGSGSGRDYTFTISSLQPED
IATYYCLQYDNLWTFGQGTKVEIKRTVAAPSVFIFPPSDEQLKSGTASVV
CLLNNFYPREAKVQWKVDNALQSGNSQESVTEQDSKDSTYSLSSTLTLSK
ADYEKHKVYACEVTHQGLSSPVTKSFNRGEC

### Natalizumab Heavy Chain

(SEQ ID NO: 3)
atgaagtgggtgaccttcatctccctgctgtttctgttctccagcgccta
ctcccaggtgcagctggtgcagtctggcgccgaagtgaagaaacctggcg
cctccgtgaaggtgtcctgcaaggcctccggcttcaacatcaaggacacc
tacatccactgggtgcgacaggcccctggccagcggctggaatggatggg
cagaatcgaccccgccaacggctacactaagtacgaccccaagttccagg
gcagagtgaccatcaccgccgacacctccgcctccaccgcctacatggaa
ctgtcctccctgcggagcgaggacaccgccgtgtactactgcgccagaga
gggctactacggcaactacggcgtgtacgccatggactactggggccagg
gcaccctggtgacagtgtcctccgccagcaccaagggcccctccgtgttc
cctctggccccttgctcccggtccacctccgagtctaccgccgctctggg
ctgcctggtgaaagactacttccccgagcccgtgaccgtgtcctggaact
ctggcgccctgacctccggcgtgcacaccttccctgccgtgctgcagtcc
tccggcctgtactccctgtcctccgtggtgaccgtgccatccagctccct
gggcaccaagacctacacctgtaacgtggaccacaagccctccaacacca
aggtggacaagcgggtggaatctaagtacggccctccctgccccagctgc
cctgcccctgaattcctgggcggaccttccgtgttcctgttccccccaaa
gcccaaggacaccctgatgatctcccggacccccgaagtgacctgcgtgg
tggtggacgtgtcccaggaagatcccgaggtgcagttcaattggtacgtg
gacggcgtggaagtgcacaacgccaagaccaagcccagagaggaacagtt
caactccacctaccgggtggtgtctgtgctgaccgtgctgcaccaggact
ggctgaacggcaaagagtacaagtgcaaggtgtccaacaagggcctgccc
agctccatcgaaaagaccatctccaaggccaagggccagccccgcgagcc
ccaggtgtacaccctgccccctagccaggaagagatgaccaagaaccagg
tgtccctgacctgtctggtgaaaggcttctacccctccgacattgccgtg
gaatgggagtccaacggccagcccgagaacaactacaagaccaccccccc
tgtgctggactccgacggctccttcttcctgtactctcggctgaccgtgg
acaagtcccggtggcaggaaggcaacgtgttctcctgctccgtgatgcac
gaggccctgcacaaccactacacccagaagtccctgtccctgagcctggg
caagtgatag

### Translation

(SEQ ID NO: 4)
MKWVTFISLLFLFSSAYSQVQLVQSGAEVKKPGASVKVSCKASGFNIKDT
YIHWVRQAPGQRLEWMGRIDPANGYTKYDPKFQGRVTITADTSASTAYME
LSSLRSEDTAVYYCAREGYYGNYGVYAMDYWGQGTLVTVSSASTKGPSVF
PLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPSC
PAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLP
SSIEKTISKAKGQPREPQVYTLPPSQEEMTKNQVSLTCLVKGFYPSDIAV
EWESNGQPENNYKTTPPVLDSDGSFFLYSRLTVDKSRWQEGNVFSCSVMH
EALHNHYTQKSLSLSLGK

### EXAMPLES

### Example 1

The initial screening of excipients was carried out using techniques that target colloidal and conformational stability of IgG₄ in various conditions using Differential Scanning Calorimetry (DSC) and Composition gradient multi-angle light scattering (CG-MALS). A sample of 20 mg/ml Natalizumab IgG₄ antibody obtained from a 10 L batch, produced at R&D DSP of Polpharma Biologic (PB006_Batch1) was used as the IgG₄ antibody.

DSC is specifically designed to determine the denaturation temperature and denaturation enthalpy of proteins and other macromolecules in solution, i.e. the amount of heat that is absorbed or released from biomolecules in solution during heating or cooling, with the versatility and precision to perform molecular stability screenings. It is a tool of choice for conformational stability. Native proteins respond to heating by unfolding (thermal denaturation) at a characteristic temperature referred to as melting temperature (Tₘ). The more intrinsically stable the biopolymer is the higher is the temperature of the unfolding transition (Tₘ) and the transition enthalpy. The experiment was performed in scanning mode in temperature range of 20° C. to 100° C. with heating and scanning rate of 1° C./min. A buffer scan was conducted prior to each sample run to generate a baseline. Samples were prepared via dialysis against the corresponding formulation buffer and diluted to about 1 mg/mL. The corresponding dialysis buffer was used as buffer scan. The exact sample concentration after dialysis was determined by UV (280 nm) absorption.

An interaction between protein molecules in solution was characterized by changes in their light scattering behavior at different concentrations via CG-MALS. CG-MALS is a well-established technique that addresses colloidal stability of the biomolecule by calculation of the second viral coefficient A₂ that describes molecule interactions in Newtonian's solutions. The characteristic parameter to measure molecule interactions is the second virial coefficient A₂. A₂ is characteristic for a macromolecule and its solvent, and describes molecular interactions between the dissolved macromolecules. A negative A₂ indicates attractive interactions whereas a positive A₂ is characteristic for repulsive interactions between the dissolved molecules.

More specifically, the CG-MALS technique is based on a concentration series of a macromolecular solution which are directly injected into the flow cell of a multi angle light scattering detector. After each injection the flow is stopped to permit the reaction to reach equilibrium. The apparent molecular weight (Mwₐₚₚ) is determined for each step in the gradient by analyzing light scattering and concentration data by the following equation:

$${Mw}_{app} \vert = \frac{R ( θ , c )}{K \times c}$$

wherein
Mwₐₚₚ: apparent molecular weight
R(θ,c): excess Rayleigh ratio of the solution as a function of scattering angle θ and concentration c. It is directly proportional to the intensity of the excess light scattered by the solute and the light scattered by the pure solvent.
K: Optical constant (4p²(dn/dc)²n₀²/NₐI₀⁴)

### c: Concentration

Significant interactions between macromolecules manifest as changes in Mwₐₚₚ vs. concentration.

A₂ calculation was conducted via Zimm plot analysis according to:

$$\frac{K \cdot c}{R ( θ , c )} = \frac{1}{{M}_{w} P ( θ )} + {A}_{2} c$$

wherein
R(θ,c): excess Rayleigh ratio of the solution as a function of scattering angle θ and concentration c. It is directly proportional to the intensity of the excess light scattered by the solute and the light scattered by the pure solvent.
Mw: Weight average molecular weight
A2: 2^{nd} virial coefficient

### c: Concentration

K: Optical constant (4p²(dn/dc)²n₀²/NₐI₀⁴)
P(θ): describes the angular dependence of the scattered light, and can be related to the rms radius. Below rms radii of 30 nm this term becomes 0.

The following selection of formulations F1-F4 were nominated for further analysis. Formulation F5 is provided for comparison and represents the current commercial formulation of natalizumab. The formulations were also evaluated for buffering capacity, conductivity and osmolality.

TABLE 1
Selected formulations based on excipients, buffers and pH screening

| | | | | | | A₂ | | |
|---|---|---|---|---|---|---|---|---|
| | | buffer | | Tₒₙₛₑₜ | Tₘₐₓ | 10⁻⁴ mol * | λ | Osmolality |
| # | pH | substance | stabilizer | ° C. | ° C. | mL * g⁻² | mS/cm | mOsm/kg |
| F1 | 5.7 | 10 mM | 150 mM | 61.6 | 81.0 | −0.102 | 15.9 | 290 |
| | | His/HCl | NaCl | | | | | |
| F2 | 6.1 | 29 mM Na- | 500 mM | 61.6 | 82.0 | −0.008 | 2.62 | 733 |
| | | phosphate | Proline | | | | | |
| F3 | 5.7 | 39 mM Na | 250 mM | 61.3 | 81.9 | −0.005 | 2.61 | 373 |
| | | acetate | Proline | | | | | |
| F4 | 6.5 | 26 mM Na- | 5% (w/v) | 65.2 | 82.3 | −0.224 | 2.63 | 340 |
| | | phosphate | Sorbitol | | | | | |
| F5 | 6.1 | 10 mM Na- | 140 mM | 62.8 | 81.4 | −0.125 | 15.8 | 281 |
| | | Phosphate | NaCl | | | | | |

### Example 2

Based on the results presented in Table 1 F1 and F4 were selected for further analysis during forced degradation studies. Formula F1 shows a good compromise between a high conductivity and A₂, and formula 4 exhibits a high Tₒₙₛₑₜ value. The requirement for conductivity of more than 2.5 mS/cm and isotonicity (250-350 mOsm/kg) were also considered during the selection of formula F1.

F1 and F4 were tested for various stresses including shaking in elevated temperature and freezing and thawing and compared to the reference formulation F5.

The shaking studies were carried at 200 rpm at 40° C. for 0.9 mL of 20 mg/ml of natalizumab batch PB006_Batch2 in 2R vials over 1 d, 3 d and 6 d. Samples were analyzed using size exclusion chromatography (SEC). SEC is a chromatographic technique that allows separation of monomeric forms from aggregated species present in biologics samples. The assay is performed using UPLC system equipped with Waters BEH200 SEC 1.7 um 4.6×150 mm at column temperature 30+/−0.5° C. The mobile phase used was 100 mM sodium phosphate buffer with 200 mM sodium chloride pH 6.8+/−0.05 at a flow rate of 0.3 ml/min. The samples were diluted to 1 mg/ml in mobile phase and then a volume of 2 μl was injected to the system. The measurement was performed at 210 nm. The SEC data after 6 days of shaking stress showed no significant differentiations between samples.

Freeze-thaw stress for liquid samples was performed in 2R vials (1.0 mL fill, 20 mg/ml of natalizumab PB006_Batch2). The samples were exposed to three cycles of freezing and thawing. Samples were frozen from room temperature to −80° C. for 8 hr and then thawed for 2 hr at room temperature. The data from DSC for the two selected formulations F1 and F4 is provided in the below table.

TABLE 2
DSC data summary. Tm1 results for selected
formulations, F1, F4 and F5.

Tₘ 1(° C.)

| | T0 | F/T |
|---|---|---|
| F1 | 67.52 | 67.8 |
| F4 | 70.46 | 70.44 |
| F5 | 68.47 | 68.56 |

The data from DSC shows conformational stability, since both formulations F1 and F4 have their Tₘ₁ (CH₂ domain unfolding) in the range from 65° C. to 70° C. In particular formulation F4 confers a slightly increased conformational stability to natalizumab.

In the second part of the experiment, liquid samples in 2R vials (1.0 mL fill, 20 mg/ml of natalizumab batch PB006_Batch2) were exposed to three cycles of freezing and thawing. Samples were frozen from room temperature to −80° C. for 8 hr and then thawed for 2 hr at room temperature. Samples were subjected to microflow imaging (MFI) before and after the three freeze-thaw-cycles.

MFI is a technique that allows characterizing sub-visible particles in the range of 2 μm-200 μm in a given formulation. Before sample preparation, the system was primed with related formulation buffer to optimize illumination first. Samples were prepared by gently mixing same in circular motions after removal from storage condition. Then the vials were opened and 1 ml of sample was withdrawn from a vial using pipette with 1 ml tip with filter. The tip, then was placed in the inlet (dispensing) port of the MFI device and tested. The sample volume of 1 ml was used to cover 0.1 ml for dead volume and 0.9 ml for dispensing. Following parameters were set on for data acquisition:

Sample dispensed: 0.9 ml
Purge volume: 0.2 ml
Sample analyzed volume: 0.59 ml
Edge particles rejection: Enabled
Fill particles: Enabled

The data was analyzed by setting data filters above 10 μm. The results are shown in the following table.

TABLE 3
MFI data summary sub-visible particle count for the range above 10
nm. Results for selected formulations, F1, F4 and F5. The data indicates
slightly increased stability for F4 and F1 formulations.

Sub-visible particles (>10 μm)

| | T0 | F/T |
|---|---|---|
| F1 | 5 | 29 |
| F4 | 23 | 13 |
| F5 | 29 | 46 |

### Example 3

In light of the foregoing results, the following formulations are tested for long term stability at various temperatures 5° C., 25° C., and 40° C., and selected time points for −70° C. The material for the study was produced at Polpharma Biologics (PB006_Batch3).

Drug Substance (DS) formulated in 10 mM Sodium Phosphate, 140 mM Sodium Chloride, 0.02% polysorbate 80, pH 6.1, was filled by Polpharma Biologics into 20 L Meissner 2D end-ported Biocontainers (TPE) (Part number: B12E00203-005) from a 250 L pilot-plant scale batch of PB006_Batch3 product, and was stored at 2-8° C. in controlled and monitored conditions until use in the stability study.

For the stability study, the material was divided into 3 equal fractions. Then the fractions were dialyzed against the respective formulation buffers that were selected for the stability studies (cf. Table 4).

TABLE 4
List of formulation buffers used in Long Term Stability studies.

| # | pH | Buffer substance | Stabilizer | Surfactant |
|---|---|---|---|---|
| F1 | 5.7 | 10 mM His/HCl | 150 mM NaCl | 0.02%(w/v) |
| | | | | Polysorbate 80 |
| F4 | 6.5 | 30 mM Na-Phosphate | 5% (w/v) Sorbitol | 0.02%(w/v) |
| | | | | Polysorbate 80 |
| F5 | 6.1 | 10 mM Na-Phosphate | 140 mM NaCl | 0.02%(w/v) |
| | | | | Polysorbate 80 |

Following buffer exchange, concentration of the samples was tested and met the target concentration of 19-21 mg/ml.

The sample vials for the stability studies were prepared under aseptic conditions in a laminal flow hood by manual filling and sealing of the sample into 15R vials. Details of primary packaging are provided in Table 5.

TABLE 5
Details of primary packaging used
in the Long Term Stability studies.

| | |
|---|---|
| Vial | 15R fiolax glass (type 1B), inner coating: baked silicon |
| Stopper | 20 mm bromobutyl rubber FM457, coated with Omniflex 3G |
| | (fluoropolymer) |
| Seal | 20 mm crimp flip-off seal of aluminum foil with a blue |
| | plastic button without embossed flip-off sign |
| Container | 15 ml (300 mg/vial) |
| Quantity | |

The packed vials were then labeled and placed under appropriate stability conditions for a period of 24-96 weeks:
Long-Term storage conditions: 5° C.±3° C.
Accelerated conditions: 25° C.±2° C.
Stress condition: 40° C.±2° C.

The program is designed for 96 weeks and data acquired during the program is analyzed by Arrhenius plots for extended shelf life calculations. The following table summarizes the sampling intervals in the program.

TABLE 6
Sampling intervals for the long term stability program

| | Storage | |
|---|---|---|
| Study | condition | Sample test interval (weeks) |
| Long-Term Storage | 5° C. ± 3° C. | 0, 1 wk, 2, 4, 6, 8, 12, 16, 20, 24, |
| Conditions | | 48, 72, 96 |
| Accelerated | 25° C. ± 2° C. | 0, 1 wk, 2, 4, 8, 12, 16, 24, 48, 96 |
| Conditions | | |
| Stress Conditions | 40° C. ± 2° C. | 0, 1 wk, 2, 4, 6, 8, 12, 16, 20, 24, |
| | | 48, 72, 96 |

Samples are tested for quality attributes using various techniques: pH measurements, total protein concentration as determined by UV-Vis, appearance (color, turbidity and visible particles), fragments including half antibody content as determined by Experion automated electrophoresis, aggregation as determined by size exclusion chromatography, sub-visible particle using Microflow Imaging (MFI), and conformational and colloidal stability. At selected time points the samples are also tested for charge variants distribution and purity by RP-LC.

Below we present data for aggregation, sub-visible particles and half-bodies content in the sample.

Size Exclusion Chromatography—is a chromatographic techniques that allows separation of monomeric forms from aggregated species present in biologics samples. The assay is performed using UPLC system equipped with Waters BEH200 SEC 1.7 um 4.6×150 mm at column temperature 30+/−0.5° C. The mobile phase used was 100 mM Sodium Phosphate buffer with 200 mM Sodium Chloride pH 6.8+/−0.05 at flow rate of 0.3 ml/min. The samples, prepared in duplicates, were diluted to 1 mg/ml in mobile phase and then a volume of 2 μl was injected to the system. The detection was performed by UV/Vis at 210 nm.

TABLE 7
SEC results for samples up to 4 weeks
% Total Aggregates by Size Exclusion Chromatography

| Time point [weeks] | 0 | 1 | 2 | 4 |
|---|---|---|---|---|
| F5_5° C. | 0.40 | 0.42 | 0.41 | 0.41 |
| F5_40° C. | 0.40 | 0.45 | 0.47 | 0.56 |
| F5_25° C. | 0.40 | 0.39 | 0.37 | 0.42 |
| F4_5° C. | 0.47 | 0.51 | 0.50 | 0.54 |
| F4_40° C. | 0.47 | 0.55 | 0.58 | 0.80 |
| F4_25° C. | 0.47 | 0.49 | 0.48 | 0.52 |
| F1_5° C. | 0.28 | 0.28 | 0.26 | 0.27 |
| F1_40° C. | 0.28 | 0.26 | 0.27 | 0.33 |
| F1_25° C. | 0.28 | 0.24 | 0.22 | 0.26 |

The data shows continuous stability of the PB006_Batch3 sample in various formulations over the period of 4 weeks at various temperatures. The long term storage and accelerated conditions show no significant increase in % aggregates indicating stability for both formulations, F1 and F4 over the time of 4 weeks.

Microflow imaging MFI is a technique that allows to characterize sub-visible particles in the range of 2 μm-200 μm present in drug product during its development.

Before sample preparation, the system was primed with related formulation buffer to optimize illumination first. Samples were prepared by gently mixing in circular motions after removal from storage condition. Then the sample preparation was performed under laminal flow hood. The vials were opened and 1 ml of sample was pipetted on a sample plate. The sample were prepared in duplicates. Then the plate was placed in the instrument autosampler. The samples tested were neat at target concentration of about 20 mg/ml. Table 8 shows the results for formulations F1, F4, and F5.

TABLE 8
MFI data for sub-visibles particles greater than 10μ
for samples up to 4 weeks in the stability program.
Sub-visible particle count by MFI

| Time point [weeks] | 0 | 1 | 2 | 4 |
|---|---|---|---|---|
| F5_5° C. | 59 | 13 | 101 | 74 |
| F5_40° C. | 59 | 7 | 6 | 4 |
| F5_25° C. | 59 | 179 | 640 | 125 |
| F4_5° C. | 202 | 24 | 14 | 80 |
| F4_40° C. | 202 | 2 | 40 | 616 |
| F4_25° C. | 202 | 807 | 154 | 765 |
| F1_5° C. | 187 | 3 | 286 | 248 |
| F1_40° C. | 187 | 8 | 22 | 17 |
| F1_25° C. | 187 | 25 | 338 | 235 |

The data shows constant stability of the PB006_Batch3 sample in three different formulations over the period of 4 weeks at 5° C., 26° C. and 40° C. The long term storage and accelerated conditions show great stability for both formulations, F1 and F4 over the time of 4 weeks.

The Experion® automated electrophoresis system (Biorad) employs LabChip microfluidic technology to automate protein electrophoresis. Separation of sample takes place on the microfluidic chip. Each chip contains a series of plastic wells bonded over a small glass plate. The glass plate is etched with a network of microchannels that connects with the base of the plastic wells. The microchannels are primed, or filled with a mixture of gel matrix and stain, before the samples are applied to the wells. Once a prepared chip is placed in the electrophoresis station and the lid is closed, 16 electrode pins contact the solutions in each well. The electrophoresis station directs the samples through the microchannels by controlling the voltages and currents that are applied. The system has sensitivity comparable to that of colloidal Coomassie Blue gel staining.

The samples were prepared by 20 fold dilution in water and in accordance with the procedure and sample preparation kit provided by the manufacturer.

TABLE 9
Data on half bodies content by chip electroforesis using Experion.
% half antibodies by chip electrophoresis (Experion ®; BioRad)

| Time point [weeks] | 0 | 1 | 2 | 4 |
|---|---|---|---|---|
| F5_5° C. | 1.53 | 1.93 | 1.75 | 1.685 |
| F5_40° C. | 1.53 | 1.46 | 1.34 | 2.24 |
| F5_25° C. | 1.53 | 1.49 | 1.19 | 1.57 |
| F4_5° C. | 1.66 | 1.61 | 1.33 | 1.56 |
| F4_40° C. | 1.66 | 1.72 | 1.23 | 2.025 |
| F4_25° C. | 1.66 | 1.36 | 1.51 | 1.77 |
| F1_5° C. | 1.730 | 0.985 | 1.080 | 1.49 |
| F1_40° C. | 1.730 | 1.485 | 1.810 | 2.895 |
| F1_25° C. | 1.730 | 1.445 | 1.180 | 1.66 |

The preliminary data for the stability program obtained using chip electrophoresis under non-reduced conditions indicates product stability in all three formulations. The level of half bodies is stable at both accelerated and long term storage conditions in all tested formulations.

The stored samples are further examined in accordance with the following table.

TABLE 10
Quality attributes and timepoints tested during the stability program.

| | Timepoints (weeks) | | | | | | | | | | | | |
|---|---|---|---|---|---|---|---|---|---|---|---|---|---|
| Quality attribute | 0 | 1 | 2 | 4 | 6 | 8 | 12 | 16 | 20 | 24 | 48 | 72 | 96 |
| Appearance (colour, visible | X | X | X | X | X | X | X | X | X | X | X | X | X |
| particles and clarity) | | | | | | | | | | | | | |
| Visual | | | | | | | | | | | | | |
| pH | X | X | X | X | X | X | X | X | X | X | X | X | X |
| Potentiometric | | | | | | | | | | | | | |
| Sub-visible particles | X | X | X | X | X | X | X | X | X | X | X | X | X |
| Light obscuration | | | | | | | | | | | | | |
| Sub-visible particles | X | X | X | X | X | X | X | X | X | X | X | X | X |
| Micro-flow imaging | | | | | | | | | | | | | |
| Amino acid sequence | X | — | — | — | — | — | — | — | — | — | — | — | — |
| UV-LC | | | | | | | | | | | | | |
| Protein concentration/Total | X | X | X | X | X | X | X | X | X | X | X | X | X |
| Protein | | | | | | | | | | | | | |
| UV280 | | | | | | | | | | | | | |
| N-Glycosylation Site & | X | — | — | — | — | — | — | — | — | — | — | — | — |
| Antibody fragments | | | | | | | | | | | | | |
| CE reduced | | | | | | | | | | | | | |
| Half antibody content & | X | X | X | X | X | X | X | X | X | X | X | X | X |
| Antibody fragments | | | | | | | | | | | | | |
| CE non-reduced | | | | | | | | | | | | | |
| Charge variants | X | X | X | X | X | X | X | X | X | X | X | X | X |
| IEX-LC | | | | | | | | | | | | | |
| Aggregation | X | X | X | X | X | X | X | X | X | X | X | X | X |
| SEC-LC | | | | | | | | | | | | | |
| Purity (imp.) | X | X | X | X | X | X | X | X | X | X | X | X | X |
| RP-LC | | | | | | | | | | | | | |
| Container Closure Integrity | X | X | X | X | X | X | X | X | X | X | X | X | X |
| Dye ingress test | | | | | | | | | | | | | |
| Binding to α4-integrin | X | — | — | X | — | X | X | X | X | X | X | X | X |
| Direct ELISA | | | | | | | | | | | | | |

Size Exclusion Chromatography is a chromatographic technique that allows separation of monomeric forms from aggregated species present in biologics samples. The assay is performed using UPLC system equipped with Waters BEH200 SEC 1.7 um 4.6×150 mm at column temperature 30+/−0.5° C. The mobile phase is 100 mM Sodium Phosphate buffer with 200 mM Sodium Chloride pH 6.8+/−0.05 at flow rate of 0.3 ml/min. The samples, prepared in duplicates, are diluted to 1 mg/ml in mobile phase and then a volume of 2 μl is injected to the system. The detection is performed by UV/Vis at 210 nm.

Monoclonal antibodies may display considerable heterogeneity that can be characterized by ion exchange liquid chromatography (IEX-LC). In a preferred embodiment, the separation is carried out by Cation Exchange (CEX) Ultra Performance Liquid Chromatography on UPLC H-Class Bio System using UV detection under Empower™ Software control. Samples are analyzed using Protein-Pak Hi Res SP (7 μm, 4.6 mm i.d.×100 mm) in a linear gradient of NaCl. Eluents are: buffer A (14 mM NaPi buffer pH 6.0) and buffer B (10.5 mM NaPi buffer pH 6.0, 0.125 M NaCl). Gradient starts with pre-equilibration of 100% buffer A in 2 min. Elution gradient starts from 10% to 30% of buffer B in 25 min, followed by a second elution step for 5 min at 30% to 60% B and re-equilibration with 90% solvent A. The total run time is 40 min. The flow rate is 0.7 ml/min. The column temperature is 40° C. and elution is monitored at 220 nm. For data evaluation Waters Empower 3 software is used. The peak assignment is performed by retention time. The sample composition is determined by detecting peaks based on their retention time and the relative proportions of each peak are calculated from the peak areas. The final results are presented as a sum of acidic species, main peak and sum of basic species.

RP-LC for antibody analysis is a chromatographic method used to detect and quantify alterations in hydrophobicity of protein fragments. Briefly, before analysis monoclonal antibody sample is digested with IdeS enzyme in order to obtain Fab and Fc fragments. Retention mechanism is based on subsequent adsorption of those fragments to stationary phase and desorption after adequate elution strength is reached. Any modifications affecting hydrophobicity, will result in Fc or Fab retention time alteration. The analyzed samples are expressed as chromatogram in which populations of different hydrophobicity are divided into peaks. Hence, RP analysis of antibody fragments method provides information concerning percentage of fragments bearing hydrophobicity-altering modifications such as methionine or tryptophan oxidation. The separation is carried out by UPLC H-Class Bio System using UV detection under Empower™ Software control. Samples are analyzed using BEH300 C4 (1.7 μm, 2.1 mm i.d.×100 mm) with linear gradient of 30→80% B for 15 minutes at flow rate of 0.2 mL/min. Mobile phases are A: 0.1% TFA in Water, B: 0.1% TFA in Acetonitrile. The column temperature is 80° C. and elution is monitored at 220 nm. For data evaluation Waters Empower 3 software is used. The peak assignment is performed by retention time. The sample composition is determined by detecting peaks based on their retention time and the relative proportions of each peak are calculated from the peak areas. The final results are presented as a sum of Fc Pre Peaks, Fc Main Peak, Fc Post Peaks, F(ab′)₂ Pre Peaks, F(ab′)₂ main peak and F(ab′)₂ Post Peaks. A sum of Fc Main Peak and F(ab′)₂ main peak is termed as ‘Purity’.

Non-reduced capillary electrophoresis (Non-red CE) is a electrophoretic method used to detect and quantify size heterogeneity or fragmentation of protein. Briefly, it involves molecule separation, based on their molecular size. The proteins form complexes with sodium dodecylsulphate that mask their native charge, so that the resulting charge is directly proportional to their molecular weight. When electric field is applied, the protein molecules are separated according to their mass. In a preferred embodiment, the separation is carried out in 30.2 cm, 50 μm ID, bare fused silica capillary and is performed for 28 min at 7.5 kV, with detection at 214 nm. The analyzed samples are expressed as electropherogram in which different molecular size populations are divided into peaks.

UV spectrophotometry (A280) is a technique based on Lambert-Beer law and is used to determine concentration of protein in solution. Spectra from 240 to 340 nm are recorded for samples diluted to about 0.5 ml/mL with PBS buffer. Protein concentration is determined using absorbance at 280 nm value diminished by absorbance at 320 nm value.

Light obscuration is a compendial method for sub-visible particle analysis. The method is based on passing the liquid sample between laser source and light-sensitive detector. Particles present in the sample block certain amount of light from a laser beam and generate ‘shadow’. Area of the shadow is converted into equivalent circular diameter of the particle. In a preferred embodiment, a sample is diluted three-fold with appropriate formulation buffer and analyzed in four replicates. The results are expressed as particle count for particles equal to or greater than 10 μm and particles to or greater than 25 μm.

Visual inspection is a compendial method for color, turbidity and particle content analysis. The method is based on European Pharmacopoeia chapters 2.9.20, 2.2.1 and 2.2.2. In a preferred embodiment Reference solutions Y are used as color standards, NTU3, NTU6, NTU18, NTU30, NTU60, NTU120, NTU200, NTU1000 are used as turbidity standards.

The results determined at time points 0, and after 3, 6, and 12 months of storage at 5° C. are shown in the following Tables 11-13.

TABLE 11
Stability data for 20 mg/ml humanized IgG₄ (natalizumab)
in formulation F1 stored at 5° C. for 12 months.

| Test | Reportable value/ | Timepoint | | | |
|---|---|---|---|---|---|
| (Method Number) | Expected Range | Start | 3 months | 6 months | 12 months |
| Colour | Not more colored | complies¹ | complies | complies | complies |
| (Ph. Eur. 2.2.2) | than Y5, B5 or BY5 | | | | |
| Visible particles | Practically free from | complies | complies | complies | complies |
| (Ph. Eur. 2.9.20) | visible particles | | | | |
| Clarity | NMT reference | complies² | complies | complies | complies |
| (Ph. Eur. 2.2.1) | suspension III | | | | |
| | or ≤18 NTU | | | | |
| pH | 5.7 ± 0.2 | 5.7 | 5.6 | 5.7 | 5.7 |
| (Ph. Eur. 2.2.3) | | | | | |
| Light obscuration | ≤6000 per container | 20 | 10 | 80 | 75 |
| (USP <787>) | for particles ≥10 μm | | | | |
| | ≤600 per container | 0 | 0 | 5 | 40 |
| | for particles ≥25 μm | | | | |
| Micro-flow imaging | 1 μm ≤ x < 2 μm | 13217 | 12513 | 6773 | 27904 |
| | 2 μm ≤ x < 10 μm | 4144 | 5741 | 2422 | 7710 |
| | 10 μm ≤ x < 25 μm | 66 | 254 | 91 | 98 |
| | 25 μm ≤ x < 100 μm | 0 | 0 | 25 | 25 |
| UV280^{c} | 18.0-22.0 mg/ml | 21.1 | 21.2 | 21.1 | 21.4 |
| CE reduced | Purity | 99.3 | — | — | — |
| | Sum of low | 0.1 | — | — | — |
| | molecular weight | | | | |
| | variants | | | | |
| CE non-reduced | Purity: ≥88.0% | 98.0 | 98.1 | 97.9 | 97.6 |
| | Sum of low | 1.7 | 1.7 | 1.9 | 1.8 |
| | molecular weights/% | | | | |
| | Sum of high | 0.2 | 0.2 | 0.2 | 0.6 |
| | molecular weights/% | | | | |
| IEX-LC | Sum of Acidic | 14.8 | 14.5 | 14.4 | 14.6 |
| | Peaks: ≤25.0% | | | | |
| | Sum of Basic Peaks | 8.6 | 9.2 | 8.6 | 10.8 |
| | Main Peak: ≥63.0% | 76.5 | 76.3 | 77.0 | 74.6 |
| SEC-LC | Dimers: ≤1.5% | 0.5 | 0.5 | 0.6 | 0.7 |
| | Higher order | 0.0 | 0.0 | 0.0 | 0.0 |
| | aggregates: ≤0.2% | | | | |
| | Aggregates³ | 0.5 | 0.5 | 0.6 | 0.7 |
| | Main Peak: ≥95.0% | 99.5 | 99.4 | 99.3 | 99.2 |
| RP-LC | Purity | 75.8 | 78.2 | 76.5 | 72.8 |
| | Sum of oxidized | 0.0 | 0.0 | 0.1 | 0.1 |
| | species^{a} | | | | |
| Binding to α4-integrin | 80-125% | not tested | — | 101.50 | 105.40 |
| ( | | | | | |

— not to be analyzed
¹Not more colored than Y5
²Clear to slightly opalescent (≤18 NTU)
³Values are sums of dimers and higher order aggregates after rounding

TABLE 12
Stability data for 20 mg/ml humanized IgG₄ (natalizumab)
in formulation F4 stored at 5° C. for 12 months.

| Test | Reportable value/ | Timepoint | | | |
|---|---|---|---|---|---|
| (Method Number) | Expected Range | Start | 3 months | 6 months | 12 months |
| Colour | Not more colored | complies¹ | complies | complies | complies |
| (Ph. Eur. 2.2.2) | than Y5, B5 or BY5 | | | | |
| Visible particles | Practically free from | complies | complies | complies | complies |
| (Ph. Eur. 2.9.20) | visible particles | | | | |
| Clarity | NMT reference | complies² | complies | complies | complies |
| (Ph. Eur. 2.2.1) | suspension III | | | | |
| | or ≤18 NTU | | | | |
| pH | 6.5 ± 0.2 | 6.6 | 6.4 | 6.5 | 6.5 |
| (Ph. Eur. 2.2.3) | | | | | |
| Light obscuration | ≤6000 per container | 5 | 15 | 70 | 23 |
| (USP <787>) | for particles ≥10 μm | | | | |
| | ≤600 per container | 0 | 0 | 30 | 0 |
| | for particles ≥25 μm | | | | |
| Micro-flow imaging | 1 μm ≤ x < 2 μm | 7789 | 18816 | 15710 | 43166 |
| | 2 μm ≤ x < 10 μm | 2916 | 6611 | 6268 | 12961 |
| | 10 μm ≤ x < 25 μm | 74 | 172 | 205 | 74 |
| | 25 μm ≤ x < 100 μm | 0 | 33 | 8 | 16 |
| UV280 | 18.0-22.0 mg/ml | 22.2 | 22.2 | 22.1 | 22.5 |
| CE reduced | Purity | 99.2 | — | — | — |
| | Sum of low | 0.1 | — | — | — |
| | molecular weight | | | | |
| | variants | | | | |
| CE non-reduced | Purity: ≥88.0% | 98.0 | 97.9 | 97.6 | 97.4 |
| | Sum of low | 1.7 | 1.8 | 2.0 | 1.7 |
| | molecular weights/% | | | | |
| | Sum of high | 0.3 | 0.3 | 0.5 | 0.8 |
| | molecular weights/% | | | | |
| IEX-LC | Sum of Acidic | 15.5 | 15.8 | 15.9 | 16.6 |
| | Peaks: ≤25.0% | | | | |
| | Sum of Basic | 8.8 | 9.1 | 8.8 | 10.2 |
| | Peaks^{a} | | | | |
| | Main Peak: ≥63.0% | 75.7 | 75.0 | 75.3 | 73.3 |
| SEC-LC | Dimers: ≤1.5% | 1.1 | 1.4 | 1.5 | 1.6 |
| | Higher order | 0.0 | 0.0 | 0.1 | 0.1 |
| | aggregates: ≤0.2% | | | | |
| | Aggregates³ | 1.1 | 1.4 | 1.6 | 1.7 |
| | Main Peak: ≥95.0% | 98.8 | 98.4 | 98.4 | 98.3 |
| RP-LC | Purity | 75.4 | 77.8 | 76.7 | 72.8 |
| ( | Sum of oxidized | 0.1 | 0.0 | 0.1 | 0.1 |
| | species | | | | |
| Binding to α4-integrin | 80-125% | not tested | — | 106.00 | 103.25 |

— not to be analyzed
¹Not more colored than Y5
²Clear to slightly opalescent (≤18 NTU)
³Values are sums of dimers and higher order aggregates after rounding

TABLE 13
Stability data for 20 mg/ml humanized IgG₄ (natalizumab)
in formulation F5 stored at 5° C. for 12 months.

| Test | Reportable value/ | Timepoint | | | |
|---|---|---|---|---|---|
| (Method Number) | Expected Range | Start | 3 months | 6 months | 12 months |
| Colour | Not more colored | complies¹ | complies | complies | complies |
| (Ph. Eur. 2.2.2) | than Y5, B5 or BY5 | | | | |
| Visible particles | Practically free from | complies | complies | complies | complies |
| (Ph. Eur. 2.9.20) | visible particles | | | | |
| Clarity | NMT reference | complies² | complies | complies | complies |
| (Ph. Eur. 2.2.1) | suspension III | | | | |
| | or ≤18 NTU | | | | |
| pH | 6.1 ± 0.2 | 6.3 | 6.2 | 6.2 | 6.3 |
| (Ph. Eur. 2.2.3) | | | | | |
| Light obscuration | ≤6000 per container | 20 | 65 | 45 | 50 |
| (USP <787>) | for particles ≥10 μm | | | | |
| | ≤600 per container | 0 | 0 | 10 | 20 |
| | for particles ≥25 μm | | | | |
| Micro-flow imaging | 1 μm ≤ x < 2 μm | 11506 | 35557 | 10060 | 28101 |
| | 2 μm ≤ x < 10 μm | 5888 | 12128 | 4269 | 8916 |
| | 10 μm ≤ x < 25 μm | 311 | 131 | 139 | 156 |
| | 25 μm ≤ x < 100 μm | 25 | 8 | 16 | 33 |
| UV280 | 18.0-22.0 mg/ml | 20.5 | 20.5 | 20.8 | 21.1 |
| CE reduced | Purity | 99.3 | — | — | — |
| | Sum of low | 0.1 | — | — | — |
| | molecular weight | | | | |
| | variants | | | | |
| CE non-reduced | Purity: ≥88.0% | 97.8 | 97.9 | 97.7 | 97.4 |
| | Sum of low | 1.7 | 1.8 | 1.9 | 1.8 |
| | molecular weights/% | | | | |
| | Sum of high | 0.5 | 0.3 | 0.4 | 0.8 |
| | molecular weights/% | | | | |
| IEX-LC | Sum of Acidic | 15.1 | 15.1 | 15.1 | 15.4 |
| | Peaks: ≤25.0% | | | | |
| | Sum of Basic | 9.0 | 8.9 | 9.0 | 10.3 |
| | Peaks^{a} | | | | |
| | Main Peak: ≥63.0% | 75.9 | 76.1 | 75.9 | 74.3 |
| SEC-LC | Dimers: ≤1.5% | 1.0 | 1.1 | 1.2 | 1.3 |
| | Higher order | 0.0 | 0.0 | 0.0 | 0.0 |
| | aggregates: ≤0.2% | | | | |
| | Aggregates³ | | | | 1.3 |
| | Main Peak: ≥95.0% | 99.0 | 98.8 | 98.7 | 98.6 |
| RP-LC | Purity | 75.8 | 78.2 | 77.4 | 72.4 |
| | Sum of oxidized | 0.0 | 0.0 | 0.1 | 0.1 |
| | species | | | | |
| Binding to α4-integrin | 80-125% | 92.00 | — | 97.40 | 101.30 |

— not to be analyzed
¹Not more colored than Y5
²Clear to slightly opalescent (≤18 NTU)
³Values are sums of dimers and higher order aggregates after rounding

The results determined at time points 0, and after 3, and 6 months of storage at 25° C. are shown in the following Tables 14-16.

TABLE 14
Stability data for 20 mg/ml humanized IgG₄ (natalizumab)
in formulation F1 stored at 25° C. for 6 months.

| Test | Reportable value/ | Timepoint | | |
|---|---|---|---|---|
| (Method Number) | Expected Range | Start | 3 months | 6 months |
| Colour | Not more colored | complies¹ | complies | complies |
| (Ph. Eur. 2.2.2) | than Y5, B5 or BY5 | | | |
| Visible particles | Practically free from | complies | complies | complies |
| (Ph. Eur. 2.9.20) | visible particles | | | |
| Clarity | NMT reference | complies² | complies | complies |
| (Ph. Eur. 2.2.1) | suspension III | | | |
| | or ≤18 NTU | | | |
| pH | 5.7 ± 0.2 | 5.7 | 5.7 | 5.6 |
| (Ph. Eur. 2.2.3) | | | | |
| Light obscuration | ≤6000 per container | 20 | 10 | 35 |
| (USP <787>) | for particles ≥10 μm | | | |
| | ≤600 per container | 0 | 0 | 5 |
| | for particles ≥25 μm | | | |
| Micro-flow imaging | 1 μm ≤ x < 2 μm | 13217 | 27166 | 27800 |
| | 2 μm ≤ x < 10 μm | 4144 | 2761 | 3457 |
| | 10 μm ≤ x < 25 μm | 66 | 74 | 115 |
| | 25 μm ≤ x < 100 μm | 0 | 0 | 57 |
| UV280 | 18.0-22.0 mg/ml | 21.1 | 21.2 | 21.2 |
| CE reduced | Purity | 99.3 | — | 97.3 |
| | Sum of low | 0.1 | — | 1.6 |
| | molecular weight | | | |
| | variants | | | |
| CE non-reduced | Purity: ≥88.0% | 98.0 | 97.2 | 96.2 |
| | Sum of low | 1.7 | 2.5 | 3.4 |
| | molecular weights/% | | | |
| | Sum of high | 0.2 | 0.2 | 0.4 |
| | molecular weights/% | | | |
| IEX-LC | Sum of Acidic | 14.8 | 16.4 | 19.4 |
| | Peaks: ≤25.0% | | | |
| | Sum of Basic | 8.6 | 10.3 | 10.9 |
| | Peaks^{a} | | | |
| | Main Peak: ≥63.0% | 76.5 | 73.3 | 69.7 |
| SEC-LC | Dimers: ≤1.5% | 0.5 | 0.7 | 0.6 |
| | Higher order | 0.0 | 0.0 | 0.0 |
| | aggregates: ≤0.2% | | | |
| | Aggregates³ | 0.5 | 0.7 | 0.6 |
| | Main Peak: ≥95.0% | 99.5 | 99.2 | 95.5 |
| RP-LC | Purity | 75.8 | 78.1 | 76.5 |
| | Sum of oxidized | 0.0 | 0.1 | 0.6 |
| | species | | | |
| Binding to α4-integrin | 80-125% | Not tested | 104.00 | 104.00 |

— not to be analyzed
¹Not more colored than Y5
²Clear to slightly opalescent (≤18 NTU)
³Values are sums of dimers and higher order aggregates after rounding

TABLE 15
Stability data for 20 mg/ml humanized IgG₄ (natalizumab)
in formulation F4 stored at 25° C. for 6 months.

| Test | Reportable value/ | Timepoint | | |
|---|---|---|---|---|
| (Method Number) | Expected Range | Start | 3 months | 6 months |
| Colour | Not more colored | complies¹ | complies | complies |
| (Ph. Eur. 2.2.2) | than Y5, B5 or BY5 | | | |
| Visible particles | Practically free from | complies | complies | complies |
| (Ph. Eur. 2.9.20) | visible particles | | | |
| Clarity | NMT reference | complies² | complies | complies |
| (Ph. Eur. 2.2.1) | suspension III | | | |
| | or ≤18 NTU | | | |
| pH | 6.5 ± 0.2 | 6.6 | 6.4 | 6.4 |
| (Ph. Eur. 2.2.3) | | | | |
| Light obscuration | ≤6000 per container | 5 | 15 | 20 |
| (USP <787>) | for particles ≥10 μm | | | |
| | ≤600 per container | 0 | 0 | 0 |
| | for particles ≥25 μm | | | |
| Micro-flow imaging | 1 μm ≤ x < 2 μm | 7789 | 23430 | 14584 |
| | 2 μm ≤ x < 10 μm | 2916 | 8856 | 4785 |
| | 10 μm ≤ x < 25 μm | 74 | 107 | 156 |
| | 25 μm ≤ x < 100 μm | 0 | 25 | 33 |
| UV280 | 18.0-22.0 mg/ml | 22.2 | 22.1 | 22.1 |
| CE reduced | Purity | 99.2 | — | 95.1 |
| | Sum of low | 0.1 | — | 3.1 |
| | molecular weight | | | |
| | variants | | | |
| CE non-reduced | Purity: ≥88.0% | 98.0 | 96.5 | 95.1 |
| | Sum of low | 1.7 | 2.9 | 3.7 |
| | molecular weights/% | | | |
| | Sum of high | 0.3 | 0.6 | 1.2 |
| | molecular weights/% | | | |
| IEX-LC | Sum of Acidic | 15.5 | 23.6 | 30.4 |
| | Peaks: ≤25.0% | | | |
| | Sum of Basic Peaks | 8.8 | 9.5 | 9.0 |
| | Main Peak: ≥63.0% | 75.7 | 66.9 | 60.5 |
| SEC-LC | Dimers: ≤1.5% | 1.1 | 1.6 | 1.9 |
| | Higher order | 0.0 | 0.1 | 0.1 |
| | aggregates: ≤0.2% | | | |
| | Aggregates³ | 1.1 | 1.7 | 2.0 |
| | Main Peak: ≥95.0% | 98.8 | 98.2 | 97.9 |
| RP-LC | Purity | 75.4 | 77.2 | 73.6 |
| | Sum of oxidized | 0.1 | 0.2 | 0.4 |
| | species | | | |
| Binding to α4-integrin | 80-125% | Not tested | 110.50 | 100.25 |

— not to be analyzed
¹Not more colored than Y5
²Clear to slightly opalescent (≤18 NTU)
³Values are sums of dimers and higher order aggregates after rounding

TABLE 16
Stability data for 20 mg/ml humanized IgG₄ (natalizumab)
in formulation F5 stored at 25° C. for 6 months.

| | | Timepoint | | |
|---|---|---|---|---|
| Test | Reportable value/ | | 3 | 6 |
| (Method Number) | Expected Range | Start | months | months |
| CE reduced | Purity | 99.3 | — | 96.3 |
| | Sum of low | 0.1 | — | 2.4 |
| | molecular weight | | | |
| | variants | | | |
| CE non-reduced | Purity: ≥88.0% | 97.8 | 97.1 | 96.2 |
| | Sum of low | 1.7 | 2.5 | 3.3 |
| | molecular weights/% | | | |
| | Sum of high | 0.5 | 0.4 | 0.5 |
| | molecular weights/% | | | |
| IEX-LC | Sum of Acidic | 15.1 | 19.2 | 23.4 |
| | Peaks: ≤25.0% | | | |
| | Sum of Basic Peaks | 9.0 | 9.3 | 9.0 |
| | Main Peak: ≥63.0% | 75.9 | 71.5 | 67.6 |
| SEC-LC | Dimers: ≤1.5% | 1.0 | 1.2 | 1.3 |
| | Higher order | 0.0 | 0.0 | 0.1 |
| | aggregates: ≤0.2% | | | |
| | Aggregates³ | 1.0 | 1.2 | 1.4 |
| | Main Peak: ≥95.0% | 99.0 | 98.7 | 98.4 |
| RP-LC | Purity | 75.8 | 78.3 | 77.0 |
| | Sum of oxidized | 0.0 | 0.2 | 0.3 |
| | species | | | |
| Binding to α4-integrin | 80-125% | Not | 103.50 | 102.50 |
| | | tested | | |

— not to be analyzed
¹ Not more colored than Y5
² Clear to slightly opalescent (≤18 NTU)
³Values are sums of dimers and higher order aggregates after rounding

The results determined at time points 0, and after 3 months of storage at 40° C. are shown in the following Tables 17-19.

TABLE 17
Stability data for 20 mg/ml humanized IgG₄ (natalizumab)
in formulation F1 stored at 40° C. for 3 months.

| Test | Reportable value/ | Timepoint | |
|---|---|---|---|
| (Method Number) | Expected Range | Start | 3 months |
| Colour | Not more colored | complies¹ | complies |
| (Ph. Eur. 2.2.2) | than Y5, B5 or BY5 | | |
| Visible particles | Practically free from | complies | complies |
| (Ph. Eur. 2.9.20) | visible particles | | |
| Clarity | NMT reference | complies² | complies |
| (Ph. Eur. 2.2.1) | suspension III | | |
| | or ≤18 NTU | | |
| pH | 5.7 ± 0.2 | 5.7 | 5.7 |
| (Ph. Eur. 2.2.3) | | | |
| Light obscuration | ≤6000 per container | 20 | 10 |
| (USP <787>) | for particles ≥10 μm | | |
| | ≤600 per container | 0 | 0 |
| | for particles ≥25 μm | | |
| Micro-flow imaging | 1 μm ≤ x < 2 μm | 13217 | 52831 |
| | 2 μm ≤ x < 10 μm | 4144 | 19071 |
| | 10 μm ≤ x < 25 μm | 66 | 426 |
| | 25 μm ≤ x < 100 μm | 0 | 8 |
| UV280 | 18.0-22.0 mg/ml | 21.1 | 21.6 |
| CE reduced | Purity | 99.3 | 94.9 |
| | Sum of low | 0.1 | 3.6 |
| | molecular weight | | |
| | variants | | |
| CE non-reduced | Purity: ≥88.0% | 98.0 | 90.5 |
| | Sum of low | 1.7 | 9.1 |
| | molecular weights/% | | |
| | Sum of high | 0.2 | 0.4 |
| | molecular weights/% | | |
| IEX-LC | Sum of Acidic | 14.8 | 36.1 |
| | Peaks: ≤25.0% | | |
| | Sum of Basic Peaks | 8.6 | 13.9 |
| | Main Peak: ≥63.0% | 76.5 | 50.0 |
| SEC-LC | Dimers: ≤1.5% | 0.5 | 1.3⁴ |
| | Higher order | 0.0 | 0.75⁵ |
| | aggregates: ≤0.2% | | |
| | Aggregates³ | 0.5 | 2.0 |
| | Main Peak: ≥95.0% | 99.5 | 95.3⁶ |
| RP-LC | Purity | 75.8 | 77.7 |
| | Sum of oxidized | 0.0 | 0.8 |
| | species | | |
| Binding to αα4-integrin | 80-125% | Not tested | 101.55 |

— not to be analyzed
¹Not more colored than Y5
²Clear to slightly opalescent (≤18 NTU)
³Values are sums of dimers and higher order aggregates after rounding
⁴Reanalysis resulted in a value of 1.4
⁵Reanalysis resulted in a value of 0.7
⁶Reanalysis resulted in a value of 95.0

TABLE 18
Stability data for 20 mg/ml humanized IgG₄ (natalizumab)
in formulation F4 stored at 40° C. for 3 months.

| Test | Reportable value/ | Timepoint | |
|---|---|---|---|
| (Method Number) | Expected Range | Start | 3 months |
| Colour | Not more colored | complies¹ | complies |
| (Ph. Eur. 2.2.2) | than Y5, B5 or BY5 | | |
| Visible particles | Practically free from | complies | complies |
| (Ph. Eur. 2.9.20) | visible particles | | |
| Clarity | NMT reference | complies² | complies |
| (Ph. Eur. 2.2.1) | suspension III | | |
| | or ≤18 NTU | | |
| pH | 6.5 ± 0.2 | 6.6 | 6.4 |
| (Ph. Eur. 2.2.3) | | | |
| Light obscuration | ≤6000 per container | 5 | 30 |
| (USP <787>) | for particles ≥10 μm | | |
| | ≤600 per container | 0 | 0 |
| | for particles ≥25 μm | | |
| Micro-flow imaging | 1 μm ≤ x < 2 μm | 7789 | 41721 |
| | 2 μm ≤ x < 10 μm | 2916 | 20754 |
| | 10 μm ≤ x < 25 μm | 74 | 1695 |
| | 25 μm ≤ x < 100 μm | 0 | 270 |
| UV280 | 18.0-22.0 mg/ml | 22.2 | 22.3 |
| CE reduced | Purity | 99.2 | 90.1 |
| | Sum of low | 0.1 | 7.2 |
| | molecular weight | | |
| | variants | | |
| CE non-reduced | Purity: ≥88.0% | 98.0 | 88.5 |
| | Sum of low | 1.7 | 10.1 |
| | molecular weights/% | | |
| | Sum of high | 0.3 | 1.4 |
| | molecular weights/% | | |
| IEX-LC | Sum of Acidic | 15.5 | 67.8 |
| | Peaks: ≤25.0% | | |
| | Sum of Basic Peaks | 8.8 | 9.7 |
| | Main Peak: ≥63.0% | 75.7 | 22.5 |
| SEC-LC | Dimers: ≤1.5% | 1.1 | 2.8 |
| | Higher order | 0.0 | 0.2 |
| | aggregates: ≤0.2% | | |
| | Aggregates³ | 1.1 | 3.0 |
| | Main Peak: ≥95.0% | 98.8 | 95.1 |
| RP-LC | Purity | 75.4 | 71.1 |
| | Sum of oxidized | 0.1 | 1.5 |
| | species | | |
| Binding to α4-integrin | 80-125% | Not tested | 100.75 |

— not to be analyzed
¹Not more colored than Y5
²Clear to slightly opalescent (≤18 NTU)
³Values are sums of dimers and higher order aggregates after rounding

TABLE 19
Stability data for 20 mg/ml humanized IgG₄ (natalizumab)
in formulation F5 stored at 40° C. for 3 months.

| Test | Reportable value/ | Timepoint | |
|---|---|---|---|
| (Method Number) | Expected Range | Start | 3 months |
| Colour | Not more colored | complies¹ | complies |
| (Ph. Eur. 2.2.2) | than Y5, B5 or BY5 | | |
| Visible particles | Practically free from | complies | complies |
| (Ph. Eur. 2.9.20) | visible particles | | |
| Clarity | NMT reference | complies² | complies |
| (Ph. Eur. 2.2.1) | suspension III | | |
| | or ≤18 NTU | | |
| pH | 6.1 ± 0.2 | 6.3 | 6.2 |
| (Ph. Eur. 2.2.3) | | | |
| Light obscuration | ≤6000 per container | 20 | 130 |
| (USP <787>) | for particles ≥10 μm | | |
| | ≤600 per container | 0 | 0 |
| | for particles ≥25 μm | | |
| Micro-flow imaging | 1 μm ≤ x < 2 μm | 11506 | 84008 |
| | 2 μm ≤ x < 10 μm | 5888 | 28694 |
| | 10 μm ≤ x < 25 μm | 311 | 1704 |
| | 25 μm ≤ x < 100 μm | 25 | 229 |
| UV280 | 18.0-22.0 mg/ml | 20.5 | 20.5 |
| CE reduced | Purity | 99.3 | 93.3 |
| | Sum of low | 0.1 | 5.1 |
| | molecular weight | | |
| | variants | | |
| CE non-reduced | Purity: ≥88.0% | 97.8 | 90.4 |
| | Sum of low | 1.7 | 9.1 |
| | molecular weights/% | | |
| | Sum of high | 0.5 | 0.4 |
| | molecular weights/% | | |
| IEX-LC | Sum of Acidic | 15.1 | 48.6 |
| | Peaks: ≤25.0% | | |
| | Sum of Basic Peaks | 9.0 | 9.7 |
| | Main Peak: ≥63.0% | 75.9 | 41.7 |
| SEC-LC | Dimers: ≤1.5% | 1.0 | 1.5 |
| | Higher order | 0.0 | 0.1 |
| | aggregates: ≤0.2% | | |
| | Aggregates³ | | |
| | Main Peak: ≥95.0% | 99.0 | 96.7 |
| RP-LC | Purity | 75.8 | 75.4 |
| | Sum of oxidized | 0.0 | 1.2 |
| | species | | |
| Binding to α4-integrin | 80-125% | Not tested | 104.50 |

— not to be analyzed
¹Not more colored than Y5
²Clear to slightly opalescent (≤18 NTU)
³Values are sums of dimers and higher order aggregates after rounding

The results show that both formulation F1 and formulation F4 exhibit at least a similar stability as compared to the prior art formulation F5. Upon storage at 5° C. and 40° C., formulation F1 shows slightly improved quality attributes as compared to formulation F5.

### Example 4

It was further decided to test additional formulation variants of formulation F1 in order to identify formulation with improved freeze-thaw stability. The following formulations of 20 mg/ml natalizumab were tested:

Formulation F1: 10 mM L-histidine, 150 mM NaCl, 0.02% (w/v) polysorbate 80, pH 5.7.
Formulation F5: 10 mM sodium phosphate, 140 mM NaCl, 0.02% polysorbate 80, pH 6.1 (control, Tysabri formulation)
Formulation F6: 10 mM L-histidine, 150 mM NaCl, 0.2% polysorbate 80, pH 5.7.
Formulation F7: 10 mM L-histidine, 20 mM NaCl, 0.02% polysorbate, pH 5.7.
Formulation F8: 10 mM L-histidine, 20 mM NaCl, 5% (w/v) sorbitol, 0.02% polysorbate 80, pH 5.7.

The antibody for the study was produced at Polpharma Biologics (PB006_Batch3). The results are provided in terms of % average content of aggregates, as determined using size exclusion chromatography. The assay is performed using UPLC system equipped with Waters BEH200 SEC 1.7 um 4.6×150 mm at column temperature 30+/−0.5° C. The mobile phase used was 100 mM sodium phosphate buffer with 200 mM sodium chloride pH 6.8+/−0.05 at a flow rate of 0.3 ml/min. The samples were diluted to 1 mg/ml in mobile phase and then a volume of 2 μl was injected to the system. The measurement was performed at 210 nm. The following table indicates the results for different methods of freeze-thawing.

TABLE 20
% Average content of aggregates for samples subjected
to different types of freeze-thaw cycles, as determined
using size exclusion chromatography.

| | F1 | F6 | F7 | F8 | F5 |
|---|---|---|---|---|---|
| Not frozen material | 0.45 | 0.47 | 0.47 | 0.47 | 0.51 |
| (1 sample) | | | | | |
| −20° C. 1 week of freezing | 0.51 | 0.75 | 0.55 | 0.49 | 0.69 |
| (average of 5 samples) | | | | | |
| −80° C. 1 week of freezing | 0.78 | 1.40 | 0.47 | 0.47 | 0.75 |
| (average of 5 samples) | | | | | |
| Liq. Nitrogen −> −80° C. | 0.90 | 1.44 | 0.47 | 0.48 | 0.69 |
| 1 week of freezing | | | | | |
| (average of 5 samples) | | | | | |

The data shown in the above table demonstrates that formulations F7 and F8 show a superior freeze-thaw stability as compared to formulations F1 (cf. Examples 2 and 3), F6, and F5 (control, Tysabri formulation).

### Example 5

The following formulations of 20 mg/ml natalizumab were additionally tested for their ability to confer stability to humanized IgG₄ such as natalizumab: Formulation F9: 10 mM L-histidine, 150 mM NaCl, 10 mM L-methionine, 0.02% (w/v) polysorbate 80, pH 5.7.

Formulation F10: 10 mM L-histidine, 150 mM NaCl, 10 mM L-methionine, 30 mM glycine, 0.02% (w/v) polysorbate 80, pH 6.1.
Formulation F11: 30 mM sodium phosphate, 5% (w/v) sucrose, 10 mM L-arginine-HCl, 0.02% polysorbate 80, pH 6.1
Formulation F12: 30 mM sodium phosphate, 5% (w/v) sucrose, 10 mM L-arginine-HCl, 2 mM L-methionine, 0.02% polysorbate 80, pH 6.1
Formulation F13: 30 mM sodium phosphate, 5% (w/v) sorbitol, 50 mM NaCl, 0.02% polysorbate 80, pH 6.5
Formulation F14: 30 mM sodium phosphate, 5% (w/v) sorbitol, 50 mM NaCl, 0.02% polysorbate 80, pH 5.7

The antibody for the study was produced at Polpharma Biologics. The formulations have been stored at 5° C., 25° C. and 40° C. for 12 weeks, and analyzed by CEX, SEC, CE non-red, ELISA, LC-MS, and DLS, as described in Example 3. The changes in the various quality attributes over storage are shown in the following tables.

TABLE 21
Formulation F9 - changes in various quality attributes after storage
for 12 weeks at 5° C., 25° C. and 40° C.

| | | Formulation F9 | | | |
|---|---|---|---|---|---|
| Parameter | | | 12 weeks | 12 weeks | 12 weeks |
| (method) | Limits | 0 weeks | @ 5° C. | @ 25° C. | @ 40° C. |
| main peak [%] | ≥63.00% | 70.76 | 70.74 | 69.71 | 36.65 |
| (CEX) | | 70.92 | | | |
| acidic species [%] | ≤25.00% | 14.11 | 13.49 | 14.52 | 46.66 |
| (CEX) | | 14.11 | | | |
| Main Peak [%] | ≥95.00% | 98.38 | 98.87 | 98.73 | 94.57 |
| (SEC) | | 98.47 | | | |
| dimers [%] | ≤1.50% | 0.36 | 0.44 | 0.56 | 1.16 |
| (SEC) | | 0.39 | | | |
| HMWI [%] | ≤0.20% | <0.1% | <0.1% | <0.1% | 0.77 |
| (SEC) | | <0.1% | | | |
| main peak [%] | ≥88.00% | 97.26 | 98.13 | 96.8 | 88.55 |
| (CE non-red) | | 97.42 | | | |
| integrin α4β1 binding [%] | 80.00-125.00% | 103.50 | | | 88.15 |
| (ELISA) | | 103.50 | | | |
| Met 255 oxidation [%] | | 4.8 | 4.2 | 4.2 | 5.0 |
| (LC-MS) | | 5.0 | | | |
| hydrodynamic radius [nm] | | 6.1 | 6.2 | 6.1 | 6.3 |
| (DLS) | | 6.1 | | | |

TABLE 22
Formulation F10 - changes in various quality attributes after storage
for 12 weeks at 5° C., 25° C. and 40° C.

| | | Formulation F10 | | | |
|---|---|---|---|---|---|
| Parameter | | | 12 weeks | 12 weeks | 12 weeks |
| (method) | Limits | 0 weeks | @ 5° C. | @ 25° C. | @ 40° C. |
| main peak [%] | ≥63.00% | 70.88 | 70.79 | 68.67 | 34.52 |
| (CEX) | | 70.86 | | | |
| acidic species [%] | ≤25.00% | 14.18 | 13.79 | 16 | 51.41 |
| (CEX) | | 14.22 | | | |
| Main Peak [%] | ≥95.00% | 98.45 | 98.87 | 98.71 | 95.36 |
| (SEC) | | 98.46 | | | |
| dimers [%] | ≤1.50% | 0.37 | 0.47 | 0.46 | 0.79 |
| (SEC) | | 0.40 | | | |
| HMWI [%] | ≤0.20% | N/D | N/D | N/D | 0.38 |
| (SEC) | | <0.1% | | | |
| main peak [%] | ≥88.00% | 97.39 | 97.73 | 96.96 | 86.8 |
| (CE non-red) | | 97.23 | | | |
| integrin α4β1 binding [%] | 80.00-125.00% | 102.00 | | | 85.85 |
| (ELISA) | | 102.00 | | | |
| Met 255 oxidation [%] | | 4.9 | 4.2 | 4.0 | 4.5 |
| (LC-MS) | | 4.6 | | | |
| hydrodynamic radius [nm] | | 6.1 | 6.2 | 6.1 | 6.1 |
| (DLS) | | 6.1 | | | |

TABLE 23
Formulation F11 - changes in various quality attributes after storage
for 12 weeks at 5° C., 25° C. and 40° C.

| | | Formulation F11 | | | |
|---|---|---|---|---|---|
| Parameter | | | 12 weeks | 12 weeks | 12 weeks |
| (method) | Limits | 0 weeks | @ 5° C. | @ 25° C. | @ 40° C. |
| main peak [%] | ≥63.00% | 70.92 | 70.38 | 55.76 | 25.29 |
| (CEX) | | 70.92 | | | 23.97 |
| acidic species [%] | ≤25.00% | 14.20 | 13.80 | 29.4 | 61.1 |
| (CEX) | | 14.05 | | | 67.67 |
| Main Peak [%] | ≥95.00% | 98.47 | 98.78 | 98.36 | 94.03 |
| (SEC) | | 98.42 | | | 59.84 |
| dimers [%] | ≤1.50% | 0.40 | 0.60 | 0.84 | 2.29 |
| (SEC) | | 0.46 | | | 5.99 |
| HMWI [%] | ≤0.20% | <0.1% | <0.1% | <0.1% | 0.70 |
| (SEC) | | <0.1% | | | 10.05 |
| main peak [%] | ≥88.00% | 97.29 | 98.08 | 96.67 | 88.75 |
| (CE non-red) | | 97.12 | | | 62.07 |
| integrin α4β1 binding [%] | 80.00-125.00% | 100.60 | | | 87.80 |
| (ELISA) | | 100.60 | | | 33.25 |
| Met 255 oxidation [%] | | 4.9 | 4.8 | 5.0 | 11.63 |
| (LC-MS) | | 4.9 | | | 13.24 |
| hydrodynamic radius [nm] | | 11.9 | 9.5 | 29.0 | 7.40 |
| (DLS) | | 11.9 | | | 24.27 |

TABLE 24
Formulation F12 - changes in various quality attributes after storage
for 12 weeks at 5° C., 25° C. and 40° C.

| | | Formulation F12 | | | |
|---|---|---|---|---|---|
| Parameter | | | 12 weeks | 12 weeks | 12 weeks |
| (method) | Limits | 0 weeks | @ 5° C. | @ 25° C. | @ 40° C. |
| main peak [%] | ≥63.00% | 70.86 | 70.5 | 65.64 | 27.98 |
| (CEX) | | 70.87 | | | |
| acidic species [%] | ≤25.00% | 14.1 | 13.83 | 18.08 | 58.66 |
| (CEX) | | 14.3 | | | |
| Main Peak [%] | ≥95.00% | 98.40 | 98.69 | 98.48 | 95.38 |
| (SEC) | | 98.39 | | | |
| dimers [%] | ≤1.50% | 0.45 | 0.66 | 0.75 | 1.47 |
| (SEC) | | 0.50 | | | |
| HMWI [%] | ≤0.20% | <0.1% | <0.1% | <0.1% | 0.42 |
| (SEC) | | <0.1% | | | |
| main peak [%] | ≥88.00% | 97.39 | 98.01 | 96.86 | 88.59 |
| (CE non-red) | | 97.32 | | | |
| inteqrin α4β1 bindinq [%] | 80.00-125.00% | 92.15 | | | 90.45 |
| (ELISA) | | 92.15 | | | |
| Met 255 oxidation [%] | | 4.8 | 4.4 | 4.4 | 5.7 |
| (LC-MS) | | 4.9 | | | |
| hydrodynamic radius [nm] | | 7.2 | 7.3 | 7.3 | 7.7 |
| (DLS) | | 7.2 | | | |

TABLE 25
Formulation F13 - changes in various quality attributes after storage
for 12 weeks at 5° C., 25° C. and 40° C.

| | | Formulation F13 | | | |
|---|---|---|---|---|---|
| Parameter | | | 12 weeks | 12 weeks | 12 weeks |
| (method) | Limits | 0 weeks | @ 5° C. | @ 25° C. | @ 40° C. |
| main peak [%] | ≥63.00% | 70.87 | 70.34 | 59.44 | 16.85 |
| (CEX) | | | | | |
| acidic species [%] | ≤25.00% | 14.09 | 13.77 | 24.35 | 71.06 |
| (CEX) | | | | | |
| Main Peak [%] | ≥95.00% | 98.31 | 98.32 | 97.92 | 92.56 |
| (SEC) | | | | | |
| dimers [%] | ≤1.50% | 0.60 | 1.02 | 1.32 | 3.45 |
| (SEC) | | | | | |
| HMWI [%] | ≤0.20% | <0.1% | <0.1% | <0.1% | 0.78 |
| (SEC) | | | | | |
| main peak [%] | ≥88.00% | 97.34 | 98.04 | 96.6 | 86.46 |
| (CE non-red) | | | | | |
| inteqrin α4β1 bindinq [%] | 80.00-125.00% | 103.50 | | | 87.80 |
| (ELISA) | | 103.50 | | | |
| Met 255 oxidation [%] | | 4.5 | 4.5 | 5.6 | 11.6 |
| (LC-MS) | | 4.5 | | | |
| hydrodynamic radius [nm] | | 7.0 | 7.1 | 7.2 | 7.3 |
| (DLS) | | 7.0 | | | |

TABLE 26
Formulation F14 - changes in various quality attributes after storage
for 12 weeks at 5° C., 25° C. and 40° C.

| | | Formulation F14 | | | |
|---|---|---|---|---|---|
| Parameter | | | 12 weeks | 12 weeks | 12 weeks |
| (method) | Limits | 0 weeks | @ 5° C. | @ 25° C. | @ 40° C. |
| main peak [%] | ≥63.00% | 70.89 | 70.57 | 68.21 | 28.34 |
| (CEX) | | 70.87 | | | |
| acidic species [%] | ≤25.00% | 14.17 | 13.54 | 15.18 | 55.62 |
| (CEX) | | 14.15 | | | |
| Main Peak [%] | ≥95.00% | 98.38 | 98.70 | 98.35 | 94.61 |
| (SEC) | | 98.39 | | | |
| dimers [%] | ≤1.50% | 0.43 | 0.67 | 0.77 | 2.12 |
| (SEC) | | 0.45 | | | |
| HMWI [%] | ≤0.20% | <0.1% | <0.1% | <0.1% | 0.58 |
| (SEC) | | <0.1% | | | |
| main peak [%] | ≥88.00% | 97.3 | 98.13 | 97.13 | 89.37 |
| (CE non-red) | | 97.21 | | | |
| integrin α4β1 binding [%] | 80.00-125.00% | 101.00 | | | 87.95 |
| (ELISA) | | 101.00 | | | |
| Met 255 oxidation [%] | | 4.5 | 4.7 | 4.7 | 7.0 |
| (LC-MS) | | 4.9 | | | |
| hydrodynamic radius [nm] | | 7.0 | 7.1 | 7.3 | 7.2 |
| (DLS) | | 7.0 | | | |

As a result, the histidine-based formulations F9 and F10 demonstrated an advantage over phosphate-based formulations and in certain parameters such as chemical and colloidal stability (% dimer and % acidic species) also over the prior art formulation F5 (cf. Tables 13, 16, and 19 above). While formulations F9 and F10 are generally comparable, formulation F9 is chemically more stable (lower percentage of acidic species variants), while formulation F10 is more colloidally stable (lower percentage of dimers and HMWI). Among the sodium phosphate based formulations, formulations F14 and F12 presented the most favorable performance. Formulation F11 demonstrated advantage over formulation F13. Formulations F11, F12. F13, and F14 often showed a comparable performance if stored for 3 months at 5° C. or 25° C. as compared to formulation F5 (cf. Tables 13, 16, and 19 above). As compared to the histidine-based formulations F9 and F10, the phosphate-based formulations F11, F12, F13, and F14 showed an increased dimer formation at 5° C., 25° C., and 40° C. In addition, formulations F11 and F13 show a high level of acidic variants (F11 at 25° C.; and F13 at 40° C.), and high methionine 225 oxidation levels (above 10%). Formulation F11 also exhibited a change in sample color and hydrodynamic radius with multimodal size distribution.

### Example 6

In light of the results of Example 5, the following formulations of 20 mg/ml natalizumab, which represent variants of formulations F1, F9 and F10 were additionally tested for their ability to confer stability to humanized IgG₄ such as natalizumab:

TABLE 27
Composition of formulations F15-F23 comprising
20 mg/ml humanized IgG₄ (natalizumab).

| | | |
|---|---|---|
| F1 | F15 | 10 mM L-histidine, 2.5 mM Na-phosphate, |
| 10 mM Histidine | | 150 mM NaCl, 0.02% PS80, pH 5.7 |
| 150 mM NaCl | F16 | 10 mM L-histidine, 2.5 mM Na-phosphate, |
| 0.02% PS80 | | 20 mM NaCl, 0.02% PS80, pH 5.7 |
| pH 5.7 | F17 | 25 mM L-histidine, 150 mM NaCl, 0.02% |
| | | PS80, pH 5.7 |
| F9 | F18 | 10 mM L-histidine, 2.5 mM Na-phosphate, |
| 10 mM Histidine | | 150 mM NaCl, 10 mM L-methionine, 0.02% |
| 150 mM NaCl | | PS80, pH 5.7 |
| 10 mM Methionine | F19 | 10 mM L-histidine, 2.5 mM Na-phosphate, |
| 0.02% PS80 | | 20 mM NaCl, 10 mM L-methionine, 0.02% |
| pH 5.7 | | PS80, pH 5.7 |
| | F20 | 25 mM L-histidine, 150 mM NaCl, 10 mM |
| | | L-methionine, 0.02% PS80, pH 5.7 |
| F10 | F21 | 10 mM L-histidine, 2.5 mM Na-phosphate, |
| 10 mM Histidine | | 150 mM NaCl, 10 mM Methionine, 30 mM |
| 150 mM NaCl | | Glycine, 0.02% PS80, pH 6.1 |
| 10 mM Methionine | F22 | 10 mM L-histidine, 2.5 mM Na-phosphate, |
| 30 mM Glycine | | 20 mM NaCl, 10 mM Methionine, 30 mM |
| 0.02% PS80 | | Glycine, 0.02% PS80, pH 6.1 |
| pH 6.1 | F23 | 25 mM L-histidine, 150 mM NaCl, 10 mM |
| | | Methionine, 30 mM Glycine, 0.02% PS80, |
| | | pH 6.1 |

The antibody for the study was produced at Polpharma Biologics. The formulations have been subjected to three freeze-thaw cycles (freezing for 22 hours at −80° C.; thawing for 2 hours at room temperature), or stored at 45° C. for up to 14 days (samples taken at day 3, 7 and 14). The samples were analyzed by CEX, SEC, CE non-red, RP, A280, and visual inspection, as described in Example 3.

Size Exclusion Chromatography is a chromatographic technique that allows separation of monomeric forms from aggregated species present in biologics samples. The assay is performed using UPLC system equipped with Waters BEH200 SEC 1.7 um 4.6×150 mm at column temperature 30+/−0.5° C. The mobile phase is 100 mM Sodium Phosphate buffer with 200 mM Sodium Chloride pH 6.8+/−0.05 at flow rate of 0.3 ml/min. The samples, prepared in duplicates, are diluted to 1 mg/ml in mobile phase and then a volume of 2 μl is injected to the system. The detection is performed by UV/Vis at 210 nm.

Monoclonal antibodies may display considerable heterogeneity that can be characterized by ion exchange liquid chromatography (IEX-LC). In a preferred embodiment, the separation is carried out by Cation Exchange (CEX) Ultra Performance Liquid Chromatography on UPLC H-Class Bio System using UV detection under Empower™ Software control. Samples are analyzed using Protein-Pak Hi Res SP (7 μm, 4.6 mm i.d.×100 mm) in a linear gradient of NaCl. Eluents are: buffer A (14 mM NaPi buffer pH 6.0) and buffer B (10.5 mM NaPi buffer pH 6.0, 0.125 M NaCl). Gradient starts with pre-equilibration of 100% buffer A in 2 min. Elution gradient starts from 10% to 30% of buffer B in 25 min, followed by a second elution step for 5 min at 30% to 60% B and re-equilibration with 90% solvent A. The total run time is 40 min. The flow rate is 0.7 ml/min. The column temperature is 40° C. and elution is monitored at 220 nm. For data evaluation Waters Empower 3 software is used. The peak assignment is performed by retention time. The sample composition is determined by detecting peaks based on their retention time and the relative proportions of each peak are calculated from the peak areas. The final results are presented as a sum of acidic species, main peak and sum of basic species.

RP-LC for antibody analysis is a chromatographic method used to detect and quantify alterations in hydrophobicity of protein fragments. Briefly, before analysis monoclonal antibody sample is digested with IdeS enzyme in order to obtain Fab and Fc fragments. Retention mechanism is based on subsequent adsorption of those fragments to stationary phase and desorption after adequate elution strength is reached. Any modifications affecting hydrophobicity, will result in Fc or Fab retention time alteration. The analyzed samples are expressed as chromatogram in which populations of different hydrophobicity are divided into peaks. Hence, RP analysis of antibody fragments method provides information concerning percentage of fragments bearing hydrophobicity-altering modifications such as methionine or tryptophan oxidation. The separation is carried out by UPLC H-Class Bio System using UV detection under Empower™ Software control. Samples are analyzed using BEH300 C4 (1.7 μm, 2.1 mm i.d.×100 mm) with linear gradient of 30→80% B for 15 minutes at flow rate of 0.2 mL/min. Mobile phases are A: 0.1% TFA in Water, B: 0.1% TFA in Acetonitrile. The column temperature is 80° C. and elution is monitored at 220 nm. For data evaluation Waters Empower 3 software is used. The peak assignment is performed by retention time. The sample composition is determined by detecting peaks based on their retention time and the relative proportions of each peak are calculated from the peak areas. The final results are presented as a sum of Fc Pre Peaks, Fc Main Peak, Fc Post Peaks, F(ab′)₂ Pre Peaks, F(ab′)₂ main peak and F(ab′)₂ Post Peaks. A sum of Fc Main Peak and F(ab′)₂ main peak is termed as ‘Purity’.

Non-reduced capillary electrophoresis (Non-red CE) is a electrophoretic method used to detect and quantify size heterogeneity or fragmentation of protein. Briefly, it involves molecule separation, based on their molecular size. The proteins form complexes with sodium dodecylsulphate that mask their native charge, so that the resulting charge is directly proportional to their molecular weight. When electric field is applied, the protein molecules are separated according to their mass. In a preferred embodiment, the separation is carried out in 30.2 cm, 50 μm ID, bare fused silica capillary and is performed for 28 min at 7.5 kV, with detection at 214 nm. The analyzed samples are expressed as electropherogram in which different molecular size populations are divided into peaks.

UV spectrophotometry (A280) is a technique based on Lambert-Beer law and is used to determine concentration of protein in solution. Spectra from 240 to 340 nm are recorded for samples diluted to about 0.5 ml/mL with PBS buffer. Protein concentration is determined using absorbance at 280 nm value diminished by absorbance at 320 nm value.

Visual inspection is a compendial method for color, turbidity and particle content analysis. The method is based on European Pharmacopoeia chapters 2.9.20, 2.2.1 and 2.2.2. In a preferred embodiment Reference solutions Y are used as color standards, NTU3, NTU6, NTU18, NTU30, NTU60, NTU120, NTU200, NTU1000 are used as turbidity standards.

All samples analyzed during Forced Degradation study were consistent with following criteria assessed by visual inspection: Practically free from visible particles (visible particles), ≤Y5 (color), ≤NTU18 (clarity). The changes in the other various quality attributes over storage are shown in the following tables.

TABLE 28
Change in IgG₄ concentration [mg/ml] in
formulationf F1, F9, F10, F5 and F15-F23.

| | | Freeze/Thaw | Thermal Stress |
|---|---|---|---|
| Formulation | T0 | After 3 cycles | 14 days at 45° C. |
| F1 | 22.08 | 22.20 | 22.24 |
| F15 | 21.78 | 21.56 | 21.61 |
| F16 | 21.42 | 21.48 | 21.64 |
| F17 | 21.02 | 21.13 | 21.23 |
| F9 | 20.43 | 20.27 | 20.55 |
| F18 | 21.02 | 21.07 | 21.07 |
| F19 | 21.02 | 21.09 | 21.17 |
| F20 | 21.55 | 21.66 | 21.77 |
| F10 | 19.94 | 19.91 | 20.25 |
| F21 | 20.66 | 20.77 | 20.87 |
| F22 | 19.81 | 19.85 | 19.92 |
| F23 | 20.85 | 20.90 | 21.05 |
| F5 | 19.53 | 19.48 | 19.69 |

TABLE 29
Change in purity (RP-LC) in formulationf F1,
F9, F10, F5 and F15-F23 expressed as %.

| | | Freeze/Thaw | Thermal Stress |
|---|---|---|---|
| Formulation | T0 | After 3 cycles | 14 days at 45° C. |
| F1 | 82.1 | 82.6 | 80.6 |
| F15 | 81.4 | 82.0 | 80.4 |
| F16 | 81.5 | 82.0 | 79.5 |
| F17 | 81.6 | 82.0 | 80.3 |
| F9 | 81.4 | 82.0 | 80.4 |
| F18 | 81.4 | 82.0 | 80.2 |
| F19 | 81.2 | 82.2 | 79.6 |
| F20 | 81.9 | 82.1 | 79.8 |
| F10 | 81.7 | 81.9 | 80.4 |
| F21 | 81.8 | 81.8 | 80.5 |
| F22 | 81.7 | 81.7 | 80.3 |
| F23 | 82.0 | 81.8 | 80.3 |
| F5 | 88.6 | 89.0 | 87.4 |

TABLE 30
Change in aggregate formation [%] induced by 1, 2, or 3 freeze-thaw cycles, as
determined by SEC-LC in formulations F1, F9, F10, F5, and F15-F23.

| | | | | | Freeze/Thaw | | | |
|---|---|---|---|---|---|---|---|---|
| | T0 | | | | After 1 cycle | | | |
| | | | | Total | | | | Total |
| | % | % | % | aggregates | % | % | % | aggregates |
| | Monomer | Dimer | HMWI | [%] | Monomer | Dimer | HMWI | [%] |
| F1 | 98.10 | 0.37 | N/D | 0.37 | 97.12 | 0.56 | <0.1 | 0.59 |
| F15 | 97.93 | 0.37 | N/D | 0.40 | 97.17 | 0.51 | <0.1 | 0.53 |
| F16 | 97.93 | 0.37 | N/D | 0.41 | 97.27 | 0.41 | N/D | 0.41 |
| F17 | 98.05 | 0.36 | N/D | 0.37 | 97.23 | 0.49 | N/D | 0.49 |
| F9 | 98.02 | 0.36 | N/D | 0.37 | 96.96 | 0.63 | <0.1 | 0.68 |
| F18 | 97.93 | 0.37 | N/D | 0.40 | 97.17 | 0.42 | N/D | 0.42 |
| F19 | 98.01 | 0.38 | N/D | 0.39 | 97.29 | 0.39 | N/D | 0.39 |
| F20 | 97.94 | 0.37 | N/D | 0.40 | 97.24 | 0.37 | N/D | 0.37 |
| F10 | 97.95 | 0.38 | N/D | 0.39 | 97.24 | 0.37 | N/D | 0.37 |
| F21 | 97.84 | 0.40 | N/D | 0.43 | 97.20 | 0.39 | N/D | 0.39 |
| F22 | 97.90 | 0.37 | N/D | 0.41 | 97.12 | 0.39 | N/D | 0.39 |
| F23 | 97.92 | 0.36 | N/D | 0.38 | 97.23 | 0.36 | N/D | 0.36 |
| F5 | 97.45 | 0.29 | N/D | 0.29 | 97.35 | 0.35 | N/D | 0.35 |
| | Freeze/Thaw | | | | | | | |
| | After 2 cycles | | | | After 3 cycles | | | |
| | | | | Total | | | | Total |
| | % | % | % | aggregates | % | % | % | aggregates |
| | Monomer | Dimer | HMWI | [%] | Monomer | Dimer | HMWI | [%] |
| F1 | 97.10 | 0.91 | 0.22 | 1.14 | 96.42 | 1.15 | 0.30 | 1.44 |
| F15 | 97.49 | 0.72 | <0.1 | 0.79 | 96.79 | 0.90 | 0.14 | 1.04 |
| F16 | 97.68 | 0.44 | N/D | 0.47 | 97.38 | 0.39 | N/D | 0.39 |
| F17 | 97.57 | 0.54 | N/D | 0.56 | 97.14 | 0.64 | N/D | 0.64 |
| F9 | 97.21 | 0.88 | <0.1 | 0.98 | 96.45 | 1.15 | 0.23 | 1.38 |
| F18 | 97.56 | 0.57 | <0.1 | 0.60 | 97.04 | 0.72 | <0.1 | 0.76 |
| F19 | 97.77 | 0.37 | N/D | 0.37 | 97.40 | 0.36 | N/D | 0.36 |
| F20 | 97.68 | 0.47 | N/D | 0.47 | 97.30 | 0.47 | N/D | 0.47 |
| F10 | 97.72 | 0.42 | N/D | 0.42 | 97.37 | 0.43 | N/D | 0.43 |
| F21 | 97.66 | 0.42 | N/D | 0.42 | 97.23 | 0.41 | N/D | 0.41 |
| F22 | 97.63 | 0.38 | N/D | 0.40 | 97.32 | 0.39 | N/D | 0.39 |
| F23 | 97.70 | 0.37 | N/D | 0.37 | 97.39 | 0.40 | N/D | 0.40 |
| F5 | 97.74 | 0.48 | N/D | 0.48 | 97.07 | 0.70 | 0.12 | 0.81 |

TABLE 31
Change in aggregate formation [%] after 3, 7, or 14 days of storage at 45° C., as
determined by SEC-LC in formulations F1, F9, F10, F5, and F15-F23.

| | | | | | Thermal Stress | | | |
|---|---|---|---|---|---|---|---|---|
| | T0 | | | | 3 days at 45° C. | | | |
| | | | | Total | | | | Total |
| | % | % | % | aggregates | % | % | % | aggregates |
| | Monomer | Dimer | HMWI | [%] | Monomer | Dimer | HMWI | [%] |
| F1 | 98.10 | 0.37 | N/D | 0.37 | 97.04 | 0.35 | N/D | 0.35 |
| F15 | 97.93 | 0.37 | N/D | 0.40 | 97.14 | 0.34 | N/D | 0.34 |
| F16 | 97.93 | 0.37 | N/D | 0.41 | 96.99 | 0.36 | N/D | 0.36 |
| F17 | 98.05 | 0.36 | N/D | 0.37 | 96.97 | 0.33 | N/D | 0.33 |
| F9 | 98.02 | 0.36 | N/D | 0.37 | 97.03 | 0.33 | N/D | 0.33 |
| F18 | 97.93 | 0.37 | N/D | 0.40 | 97.02 | 0.34 | N/D | 0.34 |
| F19 | 98.01 | 0.38 | N/D | 0.39 | 97.01 | 0.33 | N/D | 0.33 |
| F20 | 97.94 | 0.37 | N/D | 0.40 | 97.03 | 0.31 | N/D | 0.31 |
| F10 | 97.95 | 0.38 | N/D | 0.39 | 96.97 | 0.32 | N/D | 0.32 |
| F21 | 97.84 | 0.40 | N/D | 0.43 | 96.97 | 0.32 | N/D | 0.32 |
| F22 | 97.90 | 0.37 | N/D | 0.41 | 96.92 | 0.34 | N/D | 0.34 |
| F23 | 97.92 | 0.36 | N/D | 0.38 | 96.97 | 0.30 | N/D | 0.30 |
| F5 | 97.45 | 0.29 | N/D | 0.29 | 97.29 | 0.30 | N/D | 0.30 |
| | Thermal Stress | | | | | | | |
| | 7 days at 45° C. | | | | 14 days at 45° C. | | | |
| | | | | Total | | | | Total |
| | % | % | % | aggregates | % | % | % | aggregates |
| | Monomer | Dimer | HMWI | [%] | Monomer | Dimer | HMWI | [%] |
| F1 | 96.71 | 0.36 | N/D | 0.36 | 96.06 | 0.61 | 0.33 | 0.93 |
| F15 | 96.66 | 0.37 | N/D | 0.37 | 95.84 | 0.50 | 0.24 | 0.74 |
| F16 | 96.75 | 0.37 | N/D | 0.37 | 96.22 | 0.51 | 0.20 | 0.71 |
| F17 | 96.70 | 0.34 | N/D | 0.34 | 95.65 | 0.58 | 0.46 | 1.04 |
| F9 | 96.41 | 0.33 | N/D | 0.33 | 96.03 | 0.48 | 0.34 | 0.83 |
| F18 | 96.37 | 0.34 | N/D | 0.34 | 96.08 | 0.47 | 0.24 | 0.71 |
| F19 | 96.63 | 0.34 | N/D | 0.34 | 95.93 | 0.45 | 0.20 | 0.65 |
| F20 | 96.64 | 0.32 | N/D | 0.32 | 95.55 | 0.49 | 0.52 | 1.01 |
| F10 | 96.62 | 0.32 | N/D | 0.32 | 96.29 | 0.35 | N/D | 0.35 |
| F21 | 96.49 | 0.34 | N/D | 0.34 | 96.27 | 0.35 | N/D | 0.35 |
| F22 | 96.54 | 0.34 | N/D | 0.34 | 96.31 | 0.38 | N/D | 0.38 |
| F23 | 96.56 | 0.29 | N/D | 0.29 | 96.34 | 0.30 | N/D | 0.30 |
| F5 | 97.03 | 0.31 | N/D | 0.31 | 97.10 | 0.36 | N/D | 0.36 |

TABLE 32
Change in chemical stability after 3 freeze/thaw cycles, and after 3, 7, or 14 days of
storage at 45° C., as determined by CEX in formulations F1, F9, F10, F5, and F15-F23.

| | | | | Freeze/Thaw | | | Thermal Stress | | | | | | | | |
|---|---|---|---|---|---|---|---|---|---|---|---|---|---|---|---|
| | T0 | | | After 3 cycles | | | 3 days at 45° C. | | | 7 days at 45° C. | | | 14 days at 45° C. | | |
| | % AP | % MAIN | % BP | % AP | % MAIN | % BP | % AP | % MAIN | % BP | % AP | % MAIN | % BP | % AP | % MAIN | % BP |
| F1 | 13.8 | 71.9 | 14.3 | 13.6 | 72.0 | 14.4 | 12.1 | 73.4 | 14.5 | 13.8 | 71.3 | 14.9 | 24.8 | 59.8 | 15.3 |
| F15 | 13.8 | 72.1 | 14.1 | 13.6 | 72.1 | 14.3 | 12.2 | 73.3 | 14.5 | 13.8 | 71.5 | 14.7 | 24.9 | 59.9 | 15.2 |
| F16 | 13.8 | 72.2 | 14.0 | 13.7 | 72.3 | 14.0 | 12.2 | 73.5 | 14.3 | 14.0 | 71.6 | 14.4 | 26.0 | 59.0 | 15.0 |
| F17 | 13.8 | 72.0 | 14.2 | 13.7 | 72.1 | 14.1 | 12.1 | 73.6 | 14.4 | 13.7 | 71.5 | 14.8 | 24.8 | 59.8 | 15.4 |
| F9 | 13.8 | 72.0 | 14.2 | 13.6 | 72.0 | 14.4 | 12.0 | 73.7 | 14.3 | 13.6 | 71.8 | 14.6 | 24.7 | 60.2 | 15.1 |
| F18 | 13.9 | 71.8 | 14.3 | 13.7 | 72.0 | 14.3 | 12.2 | 73.5 | 14.3 | 13.9 | 71.7 | 14.3 | 25.0 | 60.1 | 14.8 |
| F19 | 13.8 | 71.9 | 14.2 | 13.8 | 72.1 | 14.1 | 12.3 | 73.5 | 14.2 | 14.2 | 71.5 | 14.3 | 25.5 | 60.0 | 14.5 |
| F20 | 13.8 | 72.0 | 14.2 | 13.8 | 72.2 | 14.1 | 12.0 | 73.6 | 14.3 | 13.6 | 71.9 | 14.5 | 24.8 | 60.2 | 15.0 |
| F10 | 13.9 | 72.0 | 14.2 | 13.9 | 72.1 | 14.0 | 12.8 | 73.1 | 14.1 | 14.9 | 71.0 | 14.1 | 26.7 | 59.3 | 14.0 |
| F21 | 13.9 | 72.0 | 14.1 | 13.9 | 72.2 | 14.0 | 12.7 | 73.3 | 14.0 | 15.2 | 70.9 | 13.9 | 26.6 | 59.5 | 13.9 |
| F22 | 13.8 | 72.1 | 14.1 | 13.9 | 72.3 | 13.9 | 13.1 | 73.0 | 13.9 | 15.7 | 70.8 | 13.5 | 29.2 | 57.5 | 13.3 |
| F23 | 13.8 | 72.1 | 14.1 | 13.9 | 72.3 | 13.9 | 12.8 | 73.2 | 14.0 | 15.2 | 71.0 | 13.9 | 27.1 | 59.0 | 13.9 |
| F5 | 13.8 | 70.0 | 16.3 | 13.6 | 70.0 | 16.4 | 15.6 | 68.2 | 16.2 | 17.0 | 66.8 | 16.2 | 28.6 | 55.3 | 16.0 |

% AP—Acidic peak content [%];
% MAIN—main peak content [%];
% BP—basic peak content [%].

TABLE 33
Change in purity (CE non-reduced; main peak area
[%]) in formulationf F1, F9, F10, F5 and F15-F23.

| | | Freeze/Thaw | Thermal Stress | | |
|---|---|---|---|---|---|
| | | After 3 | 3 days | 7 days | 14 days |
| | T0 | cycles | at 45° C. | at 45° C. | at 45° C. |
| Formulation | Purity | Purity | Purity | Purity | Purity |
| F1 | 97.8 | 97.7 | 96.9 | 95.3 | 93.5 |
| F15 | 97.8 | 97.7 | 97.0 | 95.4 | 93.4 |
| F16 | 97.8 | 97.7 | 97.0 | 95.4 | 93.2 |
| F17 | 97.8 | 97.7 | 96.8 | 95.3 | 93.3 |
| F9 | 97.8 | 97.7 | 96.9 | 95.3 | 92.7 |
| F18 | 97.7 | 97.6 | 96.9 | 95.0 | 92.2 |
| F19 | 97.7 | 97.6 | 96.8 | 94.9 | 91.7 |
| F20 | 97.7 | 97.9 | 96.8 | 94.9 | 92.1 |
| F10 | 97.8 | 97.9 | 96.8 | 94.8 | 91.8 |
| F21 | 97.8 | 97.9 | 96.8 | 94.8 | 91.5 |
| F22 | 97.8 | 97.9 | 96.5 | 94.5 | 91.0 |
| F23 | 97.7 | 97.9 | 96.6 | 94.7 | 91.1 |
| F5 | 97.6 | 98.0 | 97.5 | 96.7 | 95.4 |

Formulations F16, F17, F19, F20, F22, and F23 showed a reduced percentage of dimer formation and total aggregates if compared to F1, F9, and F10, as well as if compared to the prior art formulation F5 (cf. data in Table 30 after 2 freeze/thaw cycles and after freeze/thaw 3 cycles). Moreover, formulations F15, F16, F18, and F19, a reduced percentage of dimer formation and total aggregates after 14 days at 45° C. if compared to F1, and F9, respectively (cf. Table 31, data for storage at 40° C.). Formulation 23 showed a reduced percentage of dimer formation and total aggregates as compared to both formulation F10 and prior art formulation F5. All tested formulations showed a superior chemical stability (reduced percentage acidic peak content, and higher main peak content as determined by CEX) under thermal stress if compared to prior art formulation F5 (cf. data for 7 days and 14 days at 45° C. in Table 32).

### LIST OF REFERENCES

EP 2 202 307 A1
EP 1 314 437 A1
WO 2014/160490
WO 2013/006461
WO 2009/009523
WO 2004/071439 A1
WO 98/56418
U.S. Pat. Nos. 8,420,081 and 8,883,146
WO 95/19790
A. M. Davies & B. Sutton, “Human IgG4: a structural perspective.” Immunological Reviews 268(1): 139-159 (2015).
Silva et al., “The S228P mutation prevents in vivo and in vitro IgG4 Fab-arm exchange as demonstrated using a combination of novel quantitative immunoassays and physiological matrix preparation.” J Biol Chem. 290(9): 5462-5469 (2015).
Spiess et al. Journal of Biological Chemistry 288(37): 26583-26593 (2013).
G. Urlaub, E. Kas, A. M. Carothers, and L. A. Chasin, “Deletion of the Diploid Dihydrofolate Locus from Cultured Mammalian Cells.” Cell, 33: 405-412 (1983).
G. Urlaub, P. J. Mitchell, E. Kas, L. A. Chasin, V. L. Funanage, T. T. Myoda and J. L. Harnlin, “The Effect of Gamma Rays at the Dihydrofolate Reductase Locus: Deletions and Inversions,” Somatic Cell and Molec. Genet., 12: 555-566 (1986).
W. Zhou, C.-C. Chen, B. Buckland and J. Aunins, “Fed-Batch Culture of Recombinant NS0 Myeloma Cells with High Monoclonal Antibody Production,” Biotechnology and Bioengineering, 55(5): 783-792 (1997).

1. An aqueous pharmaceutical composition comprising 1-50 mg/ml of a human or humanized full-length IgG4 in a formulation comprising 10-25 mM histidine buffer, 130-180 mM sodium chloride, and 0.001-0.5% (w/v) of a surfactant, pH 5.1-6.5.

2. The pharmaceutical composition of claim 1, wherein said pharmaceutical composition comprises 5-50 mg/ml of said human or humanized full-length IgG4.

3. The pharmaceutical composition of claim 1, wherein the IgG4 is a humanized IgG4.

4. The pharmaceutical composition of claim 1, wherein the human or humanized full-length IgG4 is natalizumab.

5. The pharmaceutical composition of claim 1, wherein said formulation comprises 0.005-0.1% (w/v) surfactant.

6. The pharmaceutical composition of claim 1, wherein the surfactant is a nonionic surfactant.

7. The pharmaceutical composition of claim 6, wherein the nonionic surfactant is selected from polysorbate (Tween), poloxamer (Pluronic), polyethylene glycol alkyl ether (Brij), polyethylene glycol octylphenyl ether (Triton X-100), polypropylene glycol alkyl ether, glucoside alkyl ether, glycerol alkyl ester, and dodecyldimethylamine oxide.

8. The pharmaceutical composition of claim 6, wherein the nonionic surfactant is polysorbate.

9. The pharmaceutical composition of claim 1, wherein formulation (i) comprises 10-20 mM histidine buffer.

10. The pharmaceutical composition of claim 1, wherein said histidine in formulation (i) is L-histidine.

11. The pharmaceutical composition of claim 1, wherein formulation (i) comprises 140-170 mM sodium chloride.

12. The pharmaceutical composition of claim 1, wherein in formulation (i) the pH is 5.1-6.4.

13. The pharmaceutical composition of claim 1, wherein formulation (i) further comprises 5-30 mM methionine.

14. The pharmaceutical composition of claim 13, wherein the methionine is L-methionine.

15. The pharmaceutical composition of claim 13, wherein formulation (i) further comprises 5-50 mM glycine.

16. The pharmaceutical composition of claim 1, wherein formulation (i) further comprises 1-5 mM of a phosphate buffer.

17. The pharmaceutical composition of claim 16, wherein said phosphate buffer is sodium phosphate buffer.

18. The pharmaceutical composition claim 1, wherein said formulation (i) consists of 10 mM L-histidine buffer, 150 mM NaCl, and 0.02% (w/v) polysorbate 80, pH 5.7.

19. The pharmaceutical composition of claim 1, wherein said formulation (i) consists of 10 mM L-histidine buffer, 2.5 mM sodium phosphate, 150 mM NaCl, and 0.02% (w/v) polysorbate 80, pH 5.7.

20. The pharmaceutical composition of claim 1, wherein said formulation (i) consists of 25 mM L-histidine buffer, 150 mM NaCl, and 0.02% (w/v) polysorbate 80, pH 5.7.

21. The pharmaceutical composition of claim 1, wherein said formulation (i) consists of 10 mM L-histidine buffer, 150 mM NaCl, 10 mM L-methionine, and 0.02% (w/v) polysorbate 80, pH 5.7.

22. The pharmaceutical composition of claim 1, wherein said formulation (i) consists of 10 mM L-histidine buffer, 2.5 mM sodium phosphate, 150 mM NaCl, 10 mM L-methionine, and 0.02% (w/v) polysorbate 80, pH 5.7.

23. The pharmaceutical composition of claim 1, wherein said formulation (i) consists of 25 mM L-histidine buffer, 150 mM NaCl, 10 mM L-methionine, and 0.02% (w/v) polysorbate 80, pH 5.7.

24. The pharmaceutical composition of claim 1, wherein said formulation (i) consists of 10 mM L-histidine buffer, 150 mM NaCl, 10 mM L-methionine, 30 mM glycine, and 0.02% (w/v) polysorbate 80, pH 6.1.

25. The pharmaceutical composition of claim 1, wherein said formulation (i) consists of 10 mM L-histidine buffer, 2.5 mM sodium phosphate, 150 mM NaCl, 10 mM L-methionine, 30 mM glycine, and 0.02% (w/v) polysorbate 80, pH 6.1.

26. The pharmaceutical composition of claim 1, wherein said formulation (i) consists of 25 mM L-histidine buffer, 150 mM NaCl, 10 mM L-methionine, 30 mM glycine, and 0.02% (w/v) polysorbate 80, pH 6.1.

27. The pharmaceutical composition of claim 1, wherein said formulation (i) consists of 10 mM L-histidine buffer, 150 mM NaCl, and 0.2% (w/v) polysorbate 80, pH 5.7.

28-59. (canceled)

60. The pharmaceutical composition of claim 1, wherein the conductivity of said pharmaceutical composition is at least 2.5 mS/cm.

61. An article of manufacture, comprising a pharmaceutical composition according to claim 1 and instructions for use of said composition.

62-69. (canceled)
